(19) United States
(12) Patent Application Publication
(10) Pub. No.: US 2023/0076204 A1
(43) Pub. Date: Mar. 9, 2023

(54) SINGLE CHAIN TRIMER MHC CLASS II NUCLEIC ACIDS AND PROTEINS AND METHODS OF USE

(71) Applicants: Institute for Systems Biology, Seattle, WA (US); California Institute of Technology, Pasadena, CA (US)

(72) Inventors: James R. Heath, Seattle, WA (US); William Chour, San Gabriel, CA (US); Rongyu Zhang, Seattle, WA (US)

(73) Assignees: Institute for Systems Biology, Seattle, WA (US); California Institute of Technology, Pasadena, CA (US)

(21) Appl. No.: 17/800,311

(22) PCT Filed: Feb. 18, 2021
(86) PCT No.: PCT/US21/18590

§ 371 Date: Aug. 17, 2022

Related U.S. Application Data
(60) Provisional application No. 62/978,120, filed on Feb. 18, 2020.

(51) Int. Cl. C07K 14/74; C12N 15/79; C40B 40/08; C40B 40/10; G01N 33/569

(52) U.S. Cl. CPC C07K 14/70539; C12N 15/79; C40B 40/08; C40B 40/10; G01N 33/56972

(57) ABSTRACT

Peptide-major histocompatibility (MHC) Class II nucleic acids and proteins are provided. Methods of their use, for example in methods of identifying antigen-specific T cells and adoptive cell therapy, are also provided.

### CROSS REFERENCE TO RELATED APPLICATIONS

This application claims the benefit of U.S. Provisional Application No. 62/978,120, filed Feb. 18, 2020, which is incorporated by reference herein in its entirety.

### FIELD

This disclosure relates to peptide-major histocompatibility (MHC) Class II nucleic acids and proteins, and methods of their use, for example in methods of adoptive cell therapy.

### BACKGROUND

MHC class II heterodimers consist of unique α and β chains determined by separate class II HLA alleles per locus. Because every individual may have up to two unique HLA alleles per chain type per class II locus, up to four uniquely paired α/β heterodimers may be expressed by each locus. One exception is the α chain of the HLA-DR locus, which is essentially invariant in the human species, and thus for HLA-DR, there are only two potential α/β combinations, which is analogous to the situation with β2m for MHC class I. Structurally, both α and β chains of MHC class II proteins are involved in forming the β sheet and a helices of the binding groove. Taken together, the binding groove of MHC class II molecules is typically more diverse than that of MHC class I, given the various combinations by which α and β chains can come together to influence the molecular structure of the peptide binding groove.

T cell receptors (TCRs) are heterodimers consisting of an α and β chain subunit, where both chains play a role in interacting with the epitope presented by a bound peptide-MHC (pMHC) tetramer. During lymphocytic development, the TCRα and TCRβ chain genes undergo numerous processing steps, whereby the gene segments that make up each chain recombine and undergo randomized modifications, to generate mRNA transcripts encoding the finalized TCR α/β paired sequence, which defines individual T cell clonotypes from each other.

Developments in peptide processing and binding prediction algorithms have been useful in assisting with in silico identification of immunogenic epitopes. The overarching goal of these efforts has generally been to provide a pre-experimental filter of genomically processed antigen data to narrow down the list of peptide candidates for subsequent downstream experimental steps (e.g., vaccination testing or loading onto MHCs to identify TCRs). However, on the experimental side, verification of these predicted epitopes still remains an outstanding challenge.

### SUMMARY

This disclosure addresses the bottleneck problems that have hampered existing pMHC-TCR pairing technologies. Provided herein are MHC Class II SCTs and assays for the discovery of multiple TCRs from multiple peptides, e.g., a “many-to-many” approach in contrast to the “one-to-many” assays previously used.

In some embodiments, this disclosure provides nucleic acid fragment pairs including a first nucleic acid fragment and a second nucleic acid fragment that, when assembled, encode a major histocompatibility complex (MHC) Class II single chain trimer (SCT) protein, the SCT including as operably linked subunits a human leukocyte antigen (HLA) alpha chain, an HLA beta chain, and a peptide, and wherein the first nucleic acid fragment and the second nucleic acid fragment each include a portion of an assembly site in a position, that when the first nucleic acid fragment and the second nucleic acid fragment are assembled, encode an invariant region separating the HLA alpha chain and the HLA beta chain of the encoded MHC Class II SCT protein. In some examples, the assembly site is a Gibson assembly site.

In some embodiments, the nucleic acid fragment pair, when assembled, encodes a MHC Class II SCT protein including protein subunits encoded in the following order (N-terminal to C-terminal): a secretion signal, an HLA alpha chain extracellular domain, an HLA alpha chain-invariant region linker (L1), an invariant region, a peptide, a peptide-HLA beta chain linker (L2), an HLA beta chain extracellular domain, and optionally, one or more purification tags. In such embodiments, the assembly site is positioned within the invariant region. In some examples, the secretion signal is a human HLA secretion signal, a human interferon-α2 secretion signal, or a human interferon-γ secretion signal.

In other embodiments, the nucleic acid fragment pair, when assembled, encodes a MHC Class II SCT protein including protein subunits encoded in the following order (N-terminal to C-terminal): a secretion signal, a peptide, a peptide-HLA beta chain linker (L1), an HLA beta chain extracellular domain, an HLA beta-alpha chain linker (L2), an HLA alpha chain extracellular domain, and optionally, one or more purification tags. In such embodiments, the assembly site is positioned within an invariant region of the HLA alpha chain. In some examples, the secretion signal is a human HLA secretion signal, a human interferon-α2 secretion signal, or a human interferon-γ secretion signal.

In some examples, the nucleic acid fragment pair also encodes a protein including one or more purification tags. In particular examples, the purification tag is a peptide that can be biotinylated (e.g., SEQ ID NO: 11). In other examples, the purification tag is a polyhistidine peptide.

In some embodiments, the MHC Class II SCT includes an HLA-DRA*01:01 alpha chain and an HLA-DRB*01:01 beta chain. In one non-limiting example, the nucleic acid fragment pair, when assembled, has the nucleic acid sequence of SEQ ID NO: 1 and/or the encoded protein has the amino acid sequence of SEQ ID NO: 2. In another non-limiting example, the nucleic acid fragment pair, when assembled, has the nucleic acid sequence of SEQ ID NO: 3 and/or the encoded protein has the amino acid sequence of SEQ ID NO: 4.

In some embodiments, the peptide is an antigen peptide, a self peptide, or a placeholder peptide. In one example, the placeholder peptide includes the amino acid sequence of SEQ ID NO: 18. The antigen peptide may be selected from a tumor-associated peptide, a neoantigen peptide, an autoimmune peptide, a fungal peptide, a bacterial peptide, and a viral peptide.

In some embodiments, the nucleic acid fragment pair is codon-optimized for mammalian expression, for example for expression in human cells.

Also provided are nucleic acid molecules that include a disclosed assembled nucleic acid fragment pair. The assembled nucleic acid fragment pair includes the first nucleic acid fragment operably linked to the second nucleic acid fragment.

In some embodiments, the assembled nucleic acid molecule is included in a vector. In some examples, the vector is a mammalian expression vector. In one non-limiting example, the mammalian expression vector is plasmid pcDNA3.1.

Disclosed herein are human cell lines that are transformed with a vector including an assembled nucleic acid molecule described herein. In one example, the human cell line is an HEK293 cell line, such as Expi293F™ cells.

Also provided are libraries that include a plurality of the disclosed nucleic acid fragment pairs or a plurality of the assembled nucleic acid fragment pairs.

Disclosed herein are human-glycosylated MHC Class II SCT proteins. In some examples, the human-glycosylated MHC Class II SCT protein is soluble.

In some embodiments the human-glycosylated MHC Class II SCT protein includes a peptide, such as an antigen peptide, a self peptide, or a placeholder peptide. In one example, the placeholder peptide includes the amino acid sequence of SEQ ID NO: 18. The antigen peptide may be selected from a tumor-associated peptide, a neoantigen peptide, an autoimmune peptide, a fungal peptide, a bacterial peptide, and a viral peptide.

In some embodiments, the soluble human-glycosylated MHC Class II SCT protein includes an HLA alpha chain extracellular domain, an HLA alpha chain-invariant chain linker (L1), an invariant chain, a peptide, a peptide-HLA beta chain linker (L2), and an HLA beta chain extracellular domain, in N-terminal to C-terminal order. In other embodiments, the soluble human-glycosylated MHC Class II SCT protein includes a peptide, a peptide-HLA beta chain linker (Li), an HLA beta chain extracellular domain, an HLA beta-alpha chain linker (L2), and an HLA alpha chain extracellular domain. In some examples, the soluble human-glycosylated MHC Class II SCT protein also includes one or more purification tags. In particular examples, the purification tag is a peptide that can be biotinylated (e.g., SEQ ID NO: 11). In other examples, the purification tag is a polyhistidine peptide.

In some embodiments, the soluble human-glycosylated MHC Class II SCT protein is assembled as a stable multimer, such as a stable tetramer. In additional embodiments, the soluble human-glycosylated MHC Class II SCT protein is attached to a surface, a polymer (such as a bead), or a nanoparticle scaffold

Also provided are libraries including a plurality of soluble human-glycosylated MHC Class II SCT proteins or libraries including a plurality of stable multimers of soluble human-glycosylated MHC Class II SCT proteins.

Further disclosed are methods of identifying an antigen-specific CD4⁺ T cell. In some embodiments, the methods include contacting a T cell population with one or more of the disclosed soluble human glycosylated MHC Class II SCT proteins (such as one or more stable multimers of a soluble human-glycosylated MHC Class II SCT protein) and identifying a CD4⁺ T cell reactive thereto. In some examples, the methods further include determining the identity of the identified antigen-specific T cell receptor (TCR), for example, by sequencing the TCR, and producing a population of T cells (e.g., CD4⁺ T cells) expressing the identified TCR.

In some embodiments, the methods also include administering the population of T cells expressing the antigen-specific TCR to a subject in need thereof. In some examples, the subject has cancer (such as a tumor), and the TCR is reactive to an antigen from a tumor sample obtained from the subject.

The foregoing and other features of the disclosure will become more apparent from the following detailed description, which proceeds with reference to the accompanying figures.

### BRIEF DESCRIPTION OF THE DRAWINGS

FIGS. 1A-1C show Class II pMHC structures and SCT constructs. FIG. 1A shows crystal structure of a Class II pMHC, showing the peptide antigen residing in a binding pocket that is formed from both the alpha and beta chains. FIG. 1B illustrates a Class II SCT construct as reported by Zhu et al. (Eur. J. Immunol. 27:1933-1941, 1997). Linkers, purification tags, etc., are engineered designs to promote stability and to facilitate purification. FIG. 1C illustrates a Class II SCT construct as reported by Thayer et al. (Mol. Immunol., 39:861-870, 2003).

FIGS. 2A and 2B are schematic diagrams showing HLA modularity of Class II SCT designs. FIG. 2A is a linear map of a Class II SCT design as reported by Zhu et al. with a cartoon of the expressed SCT construct (right). The fragment is split for Gibson assembly within the a chain to enable modular assembly of any α/β-encoded fragment. FIG. 2B is a linear map of a Class II SCT design as reported by Thayer et al. with a cartoon of the expressed SCT construct (right). The fragment is split for Gibson assembly within the invariant chain to enable modular assembly of any α/β-encoded fragment.

FIG. 3 is a detailed map of an exemplary SCT-Z Gibson region. Top: linear map of SCT-Z design. Bottom: detailed view of the highlighted region of the top panel, showing DNA and amino acid sequences of a region including the Gibson overlap and invariant amino acids encoding a chain of DRA*01:01 (SEQ ID NOs: 5 and 6).

FIG. 4 is a detailed map of SCT-T Gibson region. Top: linear map of SCT-T design. Bottom: detailed view of the highlighted region of the top panel, showing Gibson overlap (SEQ ID NO: 7) and the restriction enzymes selected for peptide sequence ligation.

FIGS. 5A and 5B are schematic diagrams showing peptide modularity of Class II SCT designs. FIG. 5A shows peptide substitution for SCT-Z plasmids using inverse PCR. A reverse primer encoding the reverse complement codons of a target peptide is used with a universal forward primer that binds to L1. FIG. 5B shows peptide substitution for SCT-T plasmids. Two ssDNA primers encoding a target peptide are used to assemble a dsDNA construct, which is digested by Bsu36I and BspEI to be ligated into the SCT template.

FIGS. 6A and 6B show SCT protein expression and thermal stability. FIG. 6A is an image of SDS-PAGE of transfected SCTs. (+) represents positive control Class I SCT (A*02:01 with a Wilms tumor 1 (WT1) peptide, RMFPNAPYL; SEQ ID NO: 8). FIG. 6B is a graph showing thermal melting profiles of proteins expressed using SCT-T and SCT-Z templates. The negative of change in fluorescence over change in temperature is shown at each temperature. For both FIGS. 6A and 6B, ID numbers correspond to the column “well ID” in Table 1.

FIG. 7 is an image of SDS-PAGE of SCT-T and SCT-Z protein deglycosylated with PNGase F, showing that both class II SCTs display similar mass changes due to glycosylation. NR, non-reduced; R, reduced; R-PNG, reduced with PNGase F treatment.

FIG. 8 is a graph showing tetramer stimulation of influenza-specific CD4⁺ T cells. ELISA cytokine assay measuring secretion of IFN-γ, TNF-α, and IL-2. “CD4⁺ T cells” refers to influenza-specific CD4⁺ T cell line. “Tetramer” refers to SCT-T loaded with influenza peptide (PKYVKQNTLKLAT; SEQ ID NO: 9).

FIG. 9 shows class II SCT flow cytometry validation. Flow cytometry assay of SCT-T tetramers against influenza-specific CD4⁺ T cell line. SCT-T tetramers assembled with either an influenza peptide (PKYVKQNTLKLAT; SEQ ID NO: 9) or an irrelevant peptide were incubated with either influenza-specific CD4⁺ T cells (top left) or Jurkat cells transduced with a TCR specific to ELAGIGILTV (MART-1; SEQ ID NO: 10) peptide (top right). The same experiment was performed (bottom row) with BRI tetramers loaded with the influenza peptide. Percentages in the corners of each plot represent the fraction of cells associated with each quadrant.

FIG. 10 is an image of SDS-PAGE of SCT expression for structural protein epitopes of SARS-CoV-2. Lane numbers correspond with the column “well ID” in Table 2. (+) represents positive control class II SCT (A*02:01 with WT1 peptide RMFPNAPYL; SEQ ID NO: 8).

FIG. 11 is a schematic illustration of an exemplary embodiment of adoptive cell therapy (ACT). This immunotherapy method begins with extraction of tissue (1) to identify antigens (2), such as neoantigens, if the subject has a tumor. Peptide-MHC binding affinity predictions are performed (3) to identity the best peptide candidates for pMHC generation (4). Stable pMHCs are then tetramerized and used to capture antigen-specific T cells (5), whose TCRs are subsequently sequenced (6), synthesized in plasmid constructs (7), transformed into healthy T cells (8), and administered to the subject (9). Alternatively, the subject could be vaccinated with the peptide candidates (non-ACT route).

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NOs: 1 and 2 are the nucleic acid and amino acid sequences, respectively, of an exemplary assembled SCT-Z design protein.

SEQ ID NOs: 3 and 4 are the nucleic acid and amino acid sequences, respectively, of an exemplary assembled SCT-T design protein.

SEQ ID NOs: 5 and 6 are the nucleic acid and amino acid sequences, respectively, of an exemplary portion of the invariant region and assembly site for an SCT-Z design.

SEQ ID NO: 7 is the amino acid sequence of an exemplary portion of an SCT-T design.

SEQ ID NOs: 8-10 are amino acid sequences of exemplary WT1, influenza, and MART-1 peptides, respectively.

SEQ ID NO: 11 is the amino acid sequence of a purification tag that can be biotinylated by biotin ligase.

SEQ ID NOs: 12-16 are amino acid sequences of exemplary linkers.

SEQ ID NO: 17 is an exemplary SCT-T design invariant region amino acid sequence.

SEQ ID NO: 18 is the amino acid sequence of an exemplary placeholder peptide.

SEQ ID NOs: 19-23 are amino acid sequences of exemplary peptide antigens.

SEQ ID NOs: 24 and 25 are nucleic acid sequences of exemplary primers for peptide library production.

SEQ ID NOs: 26-45 are amino acid sequences of SARS-CoV-2 peptides.

SEQ ID NOs: 46-52 are amino acid sequences of influenza virus A peptides.

SEQ ID NOs: 53-55 are amino acid sequences of bacterial peptides.

SEQ ID NO: 56 is a human immunodeficiency peptide amino acid sequence.

SEQ ID NO: 57 is the amino acid sequence of a vaccinia virus peptide.

SEQ ID NOs: 58-60 are amino acid sequences of exemplary human peptides.

### DETAILED DESCRIPTION

### I. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3^{rd} Edition, Springer, 2008 (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Autologous: Refers to tissues, cells or nucleic acids taken from an individual's own tissues. For example, in an autologous transfer or transplantation of T cells, the donor and recipient are the same person. Autologous (or “autogeneic” or “autogenous”) is related to self, or originating within an organism itself.

Human leukocyte antigen (HLA): Proteins encoded by the MHC gene complex. HLAs from MHC Class II include DR, DP, and DQ genes and are highly variable, with up to hundreds of variant alleles at some loci. HLA loci are named with HLA, followed by the locus (e.g., DRB1), and a number (such as 01:01) designating a specific allele at the locus (e.g., HLA-DRB1*01:01).

Linker: A nucleic acid or amino acid sequence that connects (e.g., covalently links) two nucleic acid or amino acid segments. In some examples, linker sequences may be included to provide rotational freedom to linked polypeptide domains and thereby to promote proper domain folding and inter- and intra-domain bonding. Linkers may be native sequences (for example, those found in naturally occurring MHC Class II proteins) or may be recombinant or artificial sequences. In one non-limiting example, linker sequences include glycine-serine amino acid sequences (or a nucleic acid sequence encoding the amino acid sequence), which include varying numbers of glycine and serine residues (e.g., glycine(4)-serine).

Major histocompatibility complex (MHC) Class II: MHC Class II molecules are formed from two noncovalently associated proteins, the α chain and the β chain. The α chain comprises al and α2 domains, and the β chain comprises β1 and β2 domains. The cleft into which the antigen fits is formed by the interaction of the α1 and β1 domains. The α2 and β2 domains are transmembrane Ig-fold like domains that anchor the α and β chains into the cell membrane of the antigen presenting cell. MHC Class II complexes, when associated with antigen (and in the presence of appropriate co-stimulatory signals) stimulate CD4⁺ T cells. The primary functions of CD4⁺ T cells are to initiate the inflammatory response, to regulate other cells in the immune system, and to provide help to B cells for antibody synthesis.

Nucleic acid fragment: A nucleic acid sequence (such as a linear sequence) of any length that, when assembled with (e.g., operably linked to) at least one other nucleic acid fragment, produces a complete nucleic acid molecule. In some embodiments, assembly of at least two nucleic acid fragments produces a nucleic acid that encodes an MHC Class II SCT of the disclosure.

Operably linked: A first nucleic acid is operably linked with a second nucleic acid when the first nucleic acid is placed in a functional relationship with the second nucleic acid. For instance, a promoter is operably linked to a coding sequence if the promoter affects the transcription or expression of the coding sequence. Where necessary to join two protein coding regions, the open reading frames are aligned. Similarly, proteins (including protein subunits, domains, and/or peptides) are operably linked when they are placed in a functional relationship with one another. In some examples, the operably linked segments are in an arrangement that does not occur in nature. Linkers may be included between nucleic acid or protein segments.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

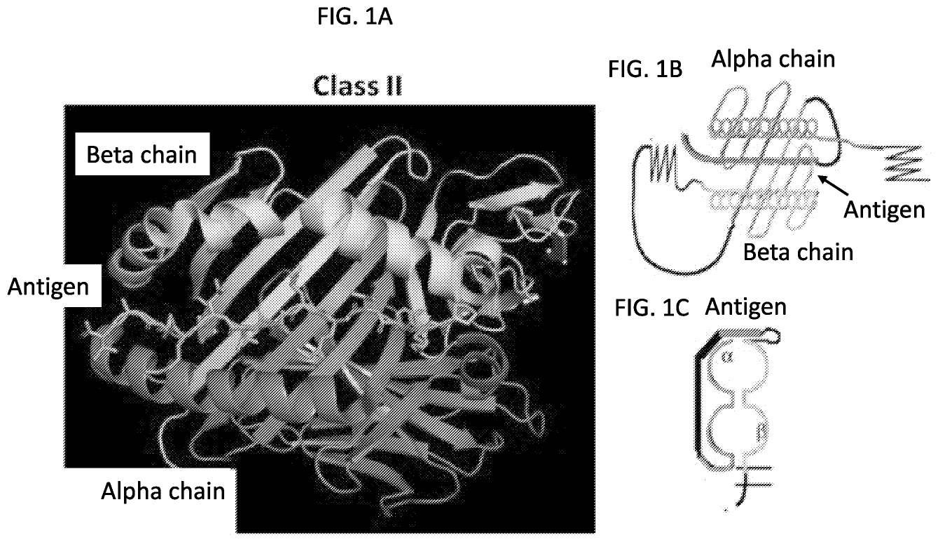

Single chain trimer (SCT): A recombinant MHC Class II molecule including all three portions of the complex (α chain, β chain, and peptide antigen) as a single, linked molecule. In some examples, SCT refers to a nucleic acid encoding an α chain, β chain, peptide antigen, and one or more linkers. In other examples, SCT refers to the protein. Two different SCT structures are schematically illustrated in FIGS. 1B and 1C.

Subject: A living multi-cellular vertebrate organism, a category that includes both human and veterinary subjects, including human and non-human mammals.

T cell: A white blood cell (lymphocyte) that is an important mediator of the immune response. T cells include, but are not limited to, CD4⁺ T cells and CD8⁺ T cells. A CD4⁺ T cell is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8⁺ T cell is a cytotoxic T lymphocyte (CTL). In another embodiment, a CD8⁺ cell is a suppressor T cell.

Activated T cells can be detected by an increase in cell proliferation and/or expression of or secretion of one or more cytokines (such as IL-2, IL-4, IL-6, IFNγ, or TNFα). Activation of CD8+ T cells can also be detected by an increase in cytolytic activity in response to an antigen.

T cell receptor (TCR): A heterodimeric protein on the surface of a T cell that binds an antigen (such as an antigen bound to an MHC molecule, for example, on an antigen presenting cell). TCRs include a and _{R} chains, each of which is a transmembrane glycoprotein. Each chain has variable and constant regions with homology to immunoglobulin variable and constant domains, a hinge region, a transmembrane domain, and a cytoplasmic tail. Similar to immunoglobulins, TCR gene segments rearrange during development to produce complete variable domains.

T cells are activated by simultaneous binding of their TCRs and co-stimulatory molecules to peptide-bound major histocompatibility complexes and complementary co-stimulatory molecules on antigen-presenting cells, respectively. For example, a CD8⁺ T cell bears T cell receptors that recognize a specific epitope when presented by a particular HLA molecule on a cell. When a CTL precursor that has been stimulated by an antigen presenting cell to become a cytotoxic T lymphocyte contacts a cell that bears such an HLA-peptide complex, the CTL forms a conjugate with the cell and destroys it.

Transduced and Transformed: A vector “transduces” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term transformation encompasses all techniques by which a nucleic acid molecule is introduced into a cell, including transformation with plasmid vectors, and introduction of naked DNA by electroporation, lipofection, and particle gun acceleration.

Treating or inhibiting a condition: “Treating” a condition refers to a therapeutic intervention that ameliorates a sign or symptom of a disease or pathological condition after it has begun to develop. “Inhibiting” refers to inhibiting the full development of the disease or condition. Inhibition of a condition can span the spectrum from partial inhibition to substantially complete inhibition of the condition. In some examples, the term “inhibiting” refers to reducing or delaying the onset or progression of a disease. A subject to be treated can be identified by standard diagnosing techniques for such a disorder, for example, based on signs and symptoms, family history, and/or risk factors to develop the disease or disorder.

Vector: A nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of an inserted gene or genes. In some non-limiting examples, the vector is a mammalian expression vector.

### II. MHC Class II SCT Nucleic Acids and Libraries

Disclosed herein are nucleic acids encoding MHC Class II SCTs and libraries including the nucleic acids. In some embodiments, the nucleic acids are provided as two or more nucleic acid fragments that when assembled encode an MHC Class II SCT. In particular examples, the SCTs are assembled from a pair of nucleic acid fragments; however, more than two nucleic acid fragments (such as 3, 4, or more) could also be utilized, by using multiple assembly sites to generate the final nucleic acid encoding the SCT.

In embodiments, provided are a nucleic acid fragment pair including a first nucleic acid fragment and second nucleic acid fragment that, when assembled, encode a major histocompatibility complex (MHC) Class II single chain trimer (SCT) protein. The SCT encoded by the assembled nucleic acid fragment pair includes as operably linked subunits a human leukocyte antigen (HLA) alpha chain, an HLA beta chain, and a peptide antigen. The first nucleic acid fragment and the second nucleic acid fragment each include a portion of an assembly site in a position, that, when the first nucleic acid fragment and the second nucleic acid fragment are assembled, encodes an invariant region separating the HLA alpha chain and the HLA beta chain of the encoded MHC Class II SCT protein. In particular examples, the assembly site is a Gibson assembly site (see, e.g., Gibson et al., Nature Methods 6:343-345, 2009). In other examples, the assembly site is a restriction enzyme site (for example, a Bsu36I restriction site).

In some embodiments, the nucleic acid fragment pair further includes a nucleic acid sequence that encodes a purification tag. In some examples, the purification tag is a polyhistidine tag (such as a 6XHis tag). In other examples, the purification tag is an amino acid sequence that can be biotinylated by biotin ligase. In one example, the purification tag encodes the amino acid sequence GLNDIFEAQKIEWHE (SEQ ID NO: 11).

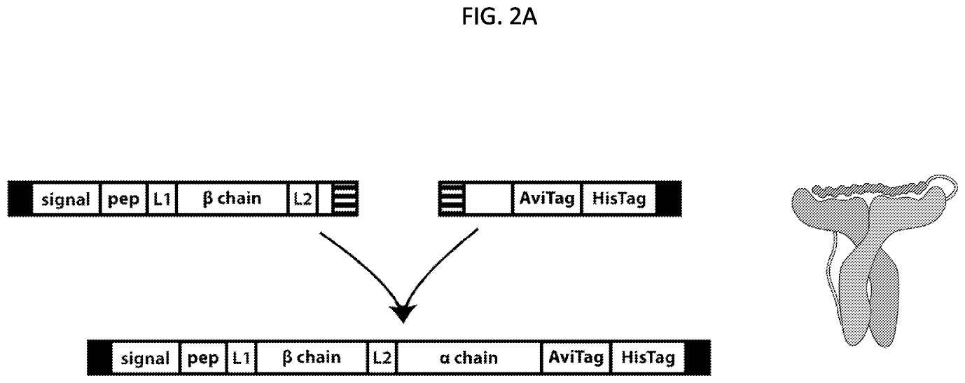

The disclosed nucleic acid fragments (such as nucleic acid fragment pairs) provide for modularity in constructions of the MHC Class II SCTs. For example, the presence of an assembly site in an invariant region of the MHC Class II SCT protein allows for modular combination of different pairs of HLA α chains and β chains. For example, by constructing nucleic acid fragments that encode an HLA α chain in one fragment and an HLA β chain in another fragment, assembled nucleic acids encoding different HLA α/β SCTs can be quickly and easily assembled. This modularity is schematically illustrated in FIGS. 2A and 2B.

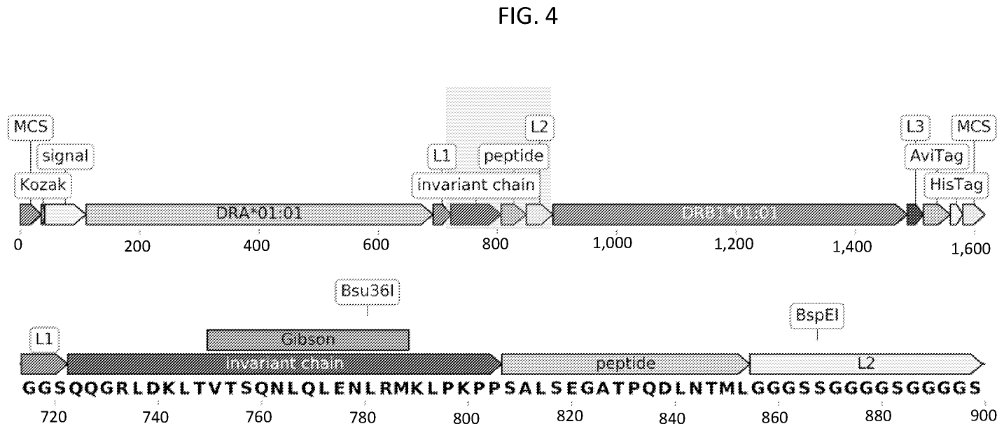

Similarly, the disclosed nucleic acid fragments (such as nucleic acid fragment pairs) also provide for modular combination of different peptides (such as different antigen peptides) with different combinations of HLA α chains and β chains. In some examples, peptide substitution is achieved by a PCR-based method, such as inverse PCR. For example, a reverse primer encoding the reverse complement of a desired peptide is used in combination with a universal forward primer (such as a universal forward primer that binds to a sequence in linker L1). This is illustrated schematically in FIG. 5A. In other examples, overlapping primers that encode a desired peptide are used to assemble a double-stranded construct including restriction enzyme recognition sites at the 5′ and 3′ ends that correspond to restriction enzyme sites flanking the peptide in the SCT template. The double-stranded construct and the SCT template are digested with the restriction enzyme(s) and ligated to produce the full-length construct.

In some embodiments, the assembled nucleic acid fragment pair encodes an SCT with what is referred to in some instances herein as a “Zhu” design SCT or “SCT-Z” (see, e.g., Zhu et al., Eur. J. Immunol. 27:1933-1941, 1997). This design encodes an SCT with protein subunits in the order (N-terminal to C-terminal): a secretion signal, a peptide (such as a peptide antigen or placeholder peptide), a first linker (L1), an HLA β chain, a second linker (L2), and an HLA α chain.

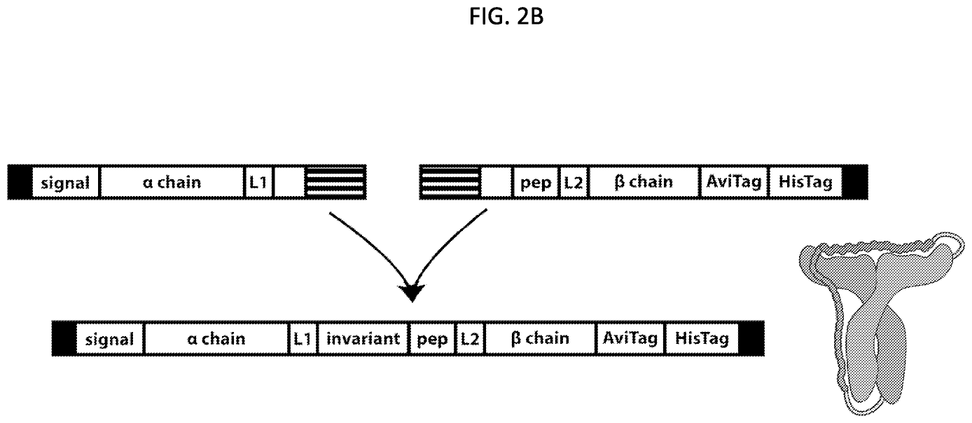

In some embodiments, the secretion signal is an HLA secretion signal (such as an HLA a secretion signal or an HLA β secretion signal). However, other secretion signals can be used, including, but not limited to a secretion signal from human interferon (IFN)-α2, human IFNγ, human interleukin-2, human serum albumin, human IgG heavy chain, or Gaussia princeps luciferase. If desired, one of ordinary skill in the art can test one or more secretion signals to identify one or more that provide increased or optimized expression levels of an SCT. In some examples, L1 encodes the amino acid sequence GGGGSLVPRGSGGGGS (SEQ ID NO: 12). In some examples, L2 encodes a glycine-serine linker, such as GGGGSGGG (SEQ ID NO: 13. In additional examples, a third linker (L3) may be included between the HLA α chain and a purification tag (if included). In some examples, L3 encodes the amino acid sequence GG. In this design, assembly sites are included in the nucleotides encoding the initial 30 amino acids of the HLA α chain. In some examples, such as HLA-DRA, HLA protein sequences are substantially invariant in this region. An exemplary assembly site is shown in FIG. 3.

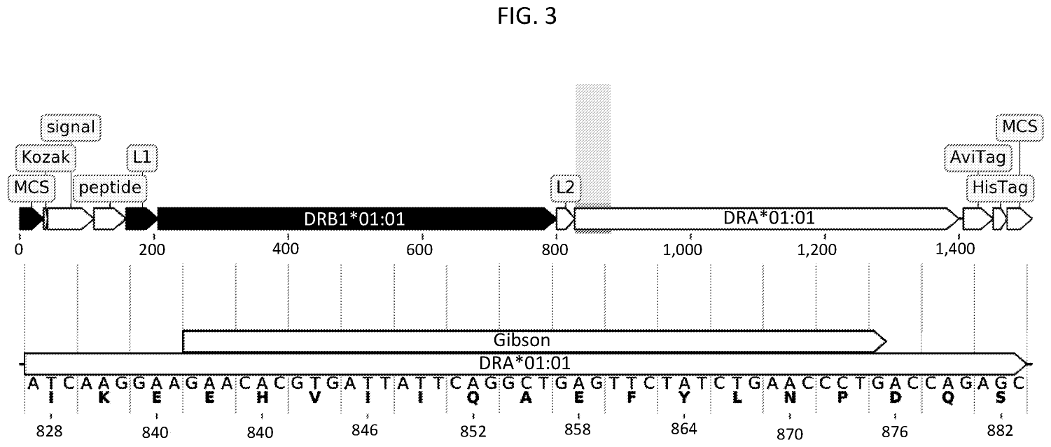

In other embodiments, the assembled nucleic acid fragment pair encodes an SCT with what is referred to in some instances herein as a “Thayer” design SCT or “SCT-T” (see, e.g., Thayer et al., Mol. Immunol. 39:861-870, 2003). This design encodes an SCT with protein subunits in the order (N-terminal to C-terminal): a secretion signal, an HLA α chain, a first linker (L1), an invariant chain, a second linker (L2), a peptide (such as a peptide antigen or placeholder peptide), and an HLA β chain. In some embodiments, the secretion signal is an HLA secretion signal (such as an HLA α secretion signal or an HLA β secretion signal). However, other secretion signals can be used, including, but not limited to a secretion signal from human interferon (IFN)-α2, human IFNγ, human interleukin-2, human serum albumin, human IgG heavy chain, or Gaussia princeps luciferase. If desired, one of ordinary skill in the art can test one or more secretion signals to identify one or more that provide increased or optimized expression levels of an SCT. In some examples, the invariant chain amino acid sequence includes or consists of QQGRLDKLTVTSQNLQLENLRMKLPKPP (SEQ ID NO: 17). In some examples, L1 encodes the amino acid sequence GGGGSGGGGS (SEQ ID NO: 14). In some examples, linker 2 (L2) encodes the amino acid sequence GGGSSGGGGSGGGGS (SEQ ID NO: 15). In additional examples, a third linker (L3) may be included between the HLA β chain and a purification tag (if included). In some examples, L3 encodes the amino acid sequence TRGGASGGG (SEQ ID NO: 16). In this design, the assembly sites are included in the nucleotides encoding the invariant chain. An exemplary assembly site is illustrated in FIG. 4.

In some embodiments, the disclosed nucleic acid fragment pairs, when assembled, encode soluble SCTs. In some embodiments, the HLA α chain is the extracellular domain of an HLA α protein. Thus, in some examples, the transmembrane domain and intracellular domain of HLA α are not included. The HLA α secretion signal may be included in the extracellular domain in some examples (for example if the HLA α chain is at the N-terminal of the SCT), while the HLA a secretion signal may be removed (for example, if the HLA α chain is internal to the SCT, or if a different secretion signal is used). In other embodiments, the HLA β chain is the extracellular domain of an HLA β protein. Thus in some examples, the transmembrane domain and intracellular domain of HLA β are not included. The HLA β secretion signal may be included in the extracellular domain in some examples (for example if the HLA chain is at the N-terminal of the SCT), while the HLA secretion signal may be removed (for example, if the HLA β chain is internal to the SCT or if a different secretion signal is used).

In other embodiments, the disclosed nucleic acid fragment pairs, when assembled, encode membrane bound SCTs. In such embodiments, the nucleic acid fragment pair further encodes a transmembrane domain and a cytoplasmic domain. Thus in some examples, the nucleic acid fragment pairs encode an HLA α chain that includes HLA α extracellular, transmembrane, and cytoplasmic domains, and an HLA β chain that includes HLA β extracellular, transmembrane, and cytoplasmic domains. See, e.g., Zhu et al., Eur. J. Immunol. 27:1933-1941, 1997; Thayer et al., Mol. Immunol. 39:861-870, 2003; Rhode et al., J. Immunol. 157:4885-4891, 1996; and Ignatowicz et al., J. Immunol. 154:3852-3862, 1995.

In some embodiments, the HLA α chain is a human HLA α chain or a mouse HLA α chain. In some examples, the human HLA α chain is selected from an HLA-DRA, HLA-DPA1, HLA-DPA2, HLA-DQA1, and HLA-DQA2 α chain. In other examples, the mouse HLA α chain is a IA-b α chain. The amino acid and nucleic acid sequences of HLA α chain alleles for each locus are publicly available, for example from EMBL-EBI (e.g., ftp.ebi.ac.uk/dpub/databases/ipd/imgt/hla/fasta/). One of ordinary skill in the art can identify other sources or sequence databases, along with updates. In some examples, the HLA α chain is included in an HLA alpha chain encoding fragment library. In particular non-limiting examples, the HLA α chain is HLA-DRA1*01:01 or HLA-DQA1*03:01.

In some embodiments, the HLA β chain is a human HLA β chain or a mouse HLA β chain. In some examples, the human HLA β chain is selected from an HLA-DRB1, HLA-DRB3, HLA-DRB4, HLA-DRBS, HLA-DPB1, HLA-DPB2, and HLA-DQB1 β chain. In other examples, the mouse HLA β chain is a IA-b β chain. The amino acid and nucleic acid sequences of HLA β chain alleles for each locus are publicly available, for example from EMBL-EBI (e.g., ftp.ebi.ac.uk/dpub/databases/ipd/imgehla/fasta/). One of ordinary skill in the art can identify other sources or sequence databases, along with updates. In some examples, the HLA β chain is included in an HLA β chain encoding fragment library. In particular non-limiting examples, the HLA β chain is DRB1*01:01, DRB1*11:04, DRB1*04:01, DRB1*15:01, or DQB1*03:02.

In some embodiments, the peptide included in the disclosed SCTs is a peptide antigen, a placeholder peptide, a self peptide (such as a peptide that occurs in healthy tissue, and is not mutated), a negative control peptide, or a positive control peptide. In some embodiments, the placeholder peptide provides “space” for the peptide-encoded region of the reverse primer to overlay (e.g., as shown in FIG. 5A), or to serve as the fragment that is removed during peptide substitution (e.g., as shown in FIG. 5B). For peptide substitution by restriction enzyme digestion, the placeholder peptide may provide spacing between enzyme cut sites to prevent or minimize spatial interference between the restriction enzymes during cleavage. Thus, in some examples, the placeholder peptide may be at least 4 amino acids long. In examples utilizing inverse PCR, a placeholder peptide may not be required, and is optional. Thus, in some examples, a placeholder peptide is from about 4-25 amino acids in length. In other examples, no placeholder peptide is present (that is, the peptide is 0 amino acids in this situation). In one example, a placeholder peptide is HIV GAG amino acids 173-188 and has the amino acid sequence SALSEGATPQDLNTML (SEQ ID NO: 18). However, other placeholder peptide sequences could be utilized, or could even be omitted in some situations, as discussed above.

In some embodiments, the peptide is a peptide antigen. A peptide antigen is a peptide that fits in the binding pocket of an MHC Class II protein complex or an MHC Class II SCT protein and is recognized by CD4⁺ T cells. In some embodiments, the peptide is about 13-25 amino acids long (e.g., 13, 14, 15, 16, 17, 18, 19, 20, 21, 22, 23, 24, 25). However, peptide antigens that are longer or shorter could also be utilized. Typically, a positive control and/or negative control peptide would be the same length as a target peptide (such as a peptide antigen), or about 13-25 amino acids long. In some examples, the peptide antigen is a tumor-associated peptide, a neoantigen peptide, an autoimmune peptide (such as a self peptide that is auto-reactive), a fungal peptide, a bacterial peptide (such as a Bacillus anthracis peptide or a Clostridium tetani peptide, for examples, SEQ ID NOs: 53-55), or a viral peptide (such as an influenza virus peptide, a coronavirus peptide, a human immunodeficiency virus (HIV) peptide, or a vaccinia virus peptide). In some examples, the peptide antigen is a viral peptide, such as an influenza A virus peptide (for example, SEQ ID NOs: 9 and 46-52), a coronavirus peptide (such as a SARS-CoV-2 peptide, for example, SEQ ID NOs: 26-45), an HIV peptide (such as SEQ ID NOs: 18, 20, and 56), or a vaccinia virus peptide (such as SEQ ID NO: 57).

Also provided herein are libraries that include a plurality of the nucleic acid fragment pairs disclosed herein. In some embodiments, the library includes 2 or more nucleic acid fragment pairs, such as 2-500 (for example, 2-50, 10-100, 20-200, 75-150, 200-400, or 300-500) nucleic acid fragment pairs. The library in some examples, includes nucleic acid fragments encoding a plurality of HLA α chains and a plurality of HLA _{R} chains and/or a plurality of peptides. Thus, in some examples, the library of nucleic acid fragment pairs can be used for modular construction of nucleic acids encoding a plurality of SCTs disclosed herein.

In some embodiments, the library includes two subsets, wherein a first subset includes a plurality of first nucleic acid fragments of the pair and a second subset includes a plurality of second nucleic acid fragments of the pair. In some examples, the first nucleic acid fragments each include at least a nucleic acid encoding an HLA α chain and the second nucleic acid fragments each include at least a nucleic acid encoding an HLA β chain. In other examples, the first nucleic acid fragments each include at least a nucleic acid encoding an HLA β chain and the second nucleic acid fragments each include at least a nucleic acid encoding at least a portion of an HLA α chain.

In some embodiments, the nucleic acid sequences encoding one or more of the SCT components of the nucleic acid fragments disclosed herein may be altered by taking advantage of the degeneracy of the genetic code such that, while the nucleotide sequence is altered, it nevertheless encodes a peptide having an amino acid sequence identical to the peptide sequences. Based upon the degeneracy of the genetic code, variant DNA molecules may be derived from the nucleic acid sequences disclosed herein or known to one of skill in the art using standard DNA mutagenesis techniques or by synthesis of DNA sequences. Thus, this disclosure also encompasses nucleic acid sequences which encode the subject SCTs, but which vary from the disclosed nucleic acid sequences by virtue of the degeneracy of the genetic code.

The nucleic acid fragments provided herein may further be codon-optimized for expression in mammalian cells. In some embodiments, the nucleic acid fragments are codon-optimized for expression in human cells. A codon-optimized nucleic acid refers to a nucleic acid sequence that has been altered such that the codons are optimal for expression in a particular system (such as a particular species or group of species). Codon optimization does not alter the amino acid sequence of the encoded protein. In some examples, codon-optimization refers to replacement of at least one codon (such as at least 5 codons, at least 10 codons, at least 25 codons, at least 50 codons, at least 75 codons, at least 100 codons or more) in a nucleic acid sequence with a synonymous codon (one that codes for the same amino acid) more frequently used (preferred) in the particular organism of interest (such as humans). Each organism has a particular codon usage bias for each amino acid, which can be determined, for example, from publicly available codon usage tables (for example see Nakamura et al., Nucleic Acids Res. 28:292, 2000). For example, a codon usage database is available on the World Wide Web at kazusa.or.jp/codon. One of skill in the art can modify a nucleic acid encoding a particular amino acid sequence, such that it encodes the same amino acid sequence, while being optimized for expression in a particular cell type (such as a human cell). Additional criteria that can be applied for codon optimization include GC content (such as average overall GC content of about 50% or about 50% GC content over given window length (such as about 30-60 bases)) and avoidance of sequences that must not be included (such as a particular restriction enzyme recognition site). In some examples, a codon-optimized sequence is generated using software, such as codon-optimization tools available from Integrated DNA Technologies (Coralville, Iowa, available on the World Wide Web at idtdna.com/CodonOpt), GenScript (Piscataway, N.J.), or Entelechon (Eurofins Genomics, Ebersberg, Germany, available on the World Wide Web at entelechon.com/2008/10/backtranslation-tool/).

Also provided are nucleic acid molecules assembled from the nucleic acid fragments (such as nucleic acid fragment pairs) disclosed herein. The assembled nucleic acid is prepared using the assembly sites present in the nucleic acid fragments. Thus, in some examples, the nucleic acid molecule is assembled by Gibson assembly. In other examples, the nucleic acid molecule is assembled by restriction enzyme digestion and ligation of the digested fragments. The assembled nucleic acid fragments are operably linked, such that the first nucleic acid fragment and second nucleic acid fragment are contiguous and the protein coding sequences are in frame.

In additional embodiments, a library including a plurality of the assembled nucleic acid molecules is also provided. In some embodiments, the library includes 2 or more such as 2-2500 (for example, 2-25, 5-50, 10-100, 20-200, 75-150, 200-400, 300-500, 400-600, 500-750, 600-800, 700-1000, 1000-1500, 1250-1750, 1500-2000, or 2000-2500) of the assembled nucleic acids. In some examples, the library of assembled nucleic acids encodes a plurality of SCTs that differ in one or more of the encoded HLA α chains, HLA β chains, and/or peptides. For example, to cover 24 HLA haplotypes, up to 48 unique fragments could be used, such that each fragment encodes either an α or a β chain. Peptides of interest can be inserted into each combination of HLA α chain and HLA β chain, as desired. In some examples, the library size of HLA α/β combinations is narrowed, for example, using an algorithm to rank peptide-HLA pairs for binding affinity. Alternatively, a single SCT HLA α/β pair is selected and a library of assembled nucleic acids is prepared, with each member having the same HLA combination, but a different peptide.

In some embodiments, the nucleic acid molecule assembled from the nucleic acid fragments (such as an assembled nucleic acid fragment pair) is included in a vector. In some examples, the vector further includes one or more expression control sequences operably linked to the assembled nucleic acid, such that expression of the assembled nucleic acid is achieved under conditions compatible with the expression control sequences. The expression control sequences include, but are not limited to, appropriate promoters, enhancers, transcription terminators, ribosome biding sequence, a start codon (e.g., ATG) 5′ of a protein-encoding nucleic acid, maintenance of the correct reading frame of that gene to permit proper translation of mRNA, and stop codons. The expression control sequence(s) in some examples are heterologous expression control sequence(s), for example from source other than the protein-encoding nucleic acid. Thus, the protein-encoding nucleic acid operably linked to a heterologous expression control sequence (such as a promoter) comprises a nucleic acid that is not naturally occurring. The vector may further include one or more additional elements, such as an origin of replication, one or more selectable marker genes (such as one or more antibiotic resistance genes), or other elements known to one of ordinary skill in the art.

Vectors for cloning, replication, and/or expression of the assembled nucleic acid molecules include bacterial plasmids, such as bacterial cloning or expression plasmids (some of which can be used for expression in bacterial and/or mammalian cells). Exemplary bacterial plasmids into which the nucleic acids can be cloned include E. coli plasmids, such as pBR322, pUC plasmids (such as pUC18 or pUC19), pBluescript, pACYC184, pCD1, pGEM® plasmids (such as pGEM®-3, pGEM®-4, pGEM-T® plasmids; Promega, Madison, Wis.), TA-cloning vectors, such as pCR® plasmids (for example, pCR® II, pCR® 2.1, or pCR® 4 plasmids; Life Technologies, Grand Island, N.Y.) or pcDNA plasmids (for example pcDNA™3.1 or pcDNA™3.3 plasmids; Life Technologies). In some examples, the vector includes a heterologous promoter which allows protein expression in bacteria. Exemplary vectors include pET vectors (for example, pET-21b), pDEST™ vectors (Life Technologies), pRSET vectors (Life Technologies), pBAD vectors, and pQE vectors (Qiagen).

In other embodiments, the vector is a mammalian expression vector. In some examples, mammalian expression vectors include a constitutive promoter, such as a CMV promoter. In other examples, the vector includes a viral origin of replication (such as an Epstein-Barr virus or SV40 origin of replication) that permits replication of the plasmid in a transformed mammalian cell. In one non-limiting example, the mammalian expression vector is a pcDNA™3 vector, for example, pcDNA™3.1 vector (ThermoFisher Scientific). However, it should be recognized that many mammalian expression vectors are available, and suitable alternatives can be selected by one of ordinary skill in the art.

Also provided are host cells, such as mammalian cells, that are transformed with a vector including an assembled nucleic acid molecule encoding an MHC Class II SCT. As utilized herein, the term “host cell” also includes any progeny of the subject host cell. Methods of transient expression or stable transfer, meaning that the foreign DNA is continuously maintained in the host, are known in the art. Techniques for the propagation of mammalian cells in culture are known to one of ordinary skill in the art. Examples of commonly used mammalian host cell lines are HEK293 cells, VERO cells, HeLa cells, CHO cells, WI38 cells, BHK cells, and COS cell lines, although other cell lines may be used, such as cells designed to provide improved expression, desirable glycosylation patterns, or other features. In some non-limiting examples, the mammalian host cells are HEK293 cells, such as Expi293F™ cells (ThermoFisher Scientific). Transformation of a host cell with recombinant DNA can be carried out by techniques known to those skilled in the art. When the host is a eukaryote, methods including transfection of DNA as calcium phosphate coprecipitates, mechanical procedures such as microinjection, electroporation, insertion of a plasmid encased in liposomes, or viral vectors can be used.

### III. Human SCT Proteins

Disclosed herein are human MHC Class II single chain trimer proteins, such as those encoded by the nucleic acid fragment pairs and assembled nucleic acids described above. As discussed in Section II, in some embodiments, mammalian host cells transformed with nucleic acid(s) encoding the disclosed SCTs are provided. In some embodiments, the human MHC Class II SCTs are soluble. In addition, as a result of expression in mammalian cells (for example, in contrast to bacterial or insect cells), the SCTs may include post-translational modifications representative of pMHCs expressed in human cells and/or are properly folded and generate functional proteins, for example at higher efficiency than those produced in non-mammalian systems. In particular embodiments, the SCTs are glycosylated.

Any of the SCT designs encoded by the nucleic acid fragment pairs or assembled nucleic acids described in Section II can be produced as soluble human glycosylated MHC Class II SCTs. Thus, in some embodiments, the soluble human glycosylated MHC Class II SCT has the organization of an HLA alpha chain, an HLA alpha chain-invariant chain linker (L1), an invariant chain, a peptide, a peptide-HLA beta chain linker (L2), and an HLA beta chain, in N-terminal to C-terminal order; or has the organization of a peptide, a peptide-HLA beta chain linker (L1), an HLA beta chain, an HLA beta-alpha chain linker (L2), and an HLA alpha chain in N-terminal to C-terminal order. The SCT may also include a purification tag. In some examples, the peptide is an antigen peptide or a placeholder peptide. In some examples, the antigen peptide is selected from a tumor-associated peptide, a neoantigen peptide, an autoimmune peptide, a fungal peptide, a bacterial peptide, and a viral peptide.

In some embodiments, soluble human-glycosylated MHC Class II SCT proteins are assembled as a stable multimer. In particular examples, the soluble human-glycosylated MHC Class II SCT proteins are assembled as stable tetramers. In some embodiments, assembly of stable multimers (such as tetramers) is carried out using biotinylated SCTs.

In one example, biotinylated SCT monomers are tetramerized with fluorophore-labeled streptavidin (such as streptavidin-phycoerythrin). In other examples, biotinylated SCT monomers are tetramerized using a custom streptavidin-DNA conjugate that allows for subsequent binding to complementary ssDNA-biotin molecules, for example affixed to streptavidin-coated beads. In a further example, SCT monomers are conjugated onto 10X-compatible DNA barcoded dextramers. These dextramers may also be labeled with fluorophores and therefore may be used after SCT conjugation in the same manner for flow cytometry as SCT-tetramers described above. Also provided are libraries of the soluble human-glycosylated MHC Class II SCT proteins, as monomers or stable multimers (such as tetramers). In some embodiments, the library includes 2 or more, such as 2-2500 (for example, 2-25, 5-50, 10-100, 20-200, 75-150, 200-400, 300-500, 400-600, 500-750, 600-800, 700-1000, 1000-1500, 1250-1750, 1500-2000, or 2000-2500) soluble human-glycosylated MHC Class II SCT proteins. In some examples, the library of soluble human-glycosylated MHC Class II SCT proteins includes a plurality of SCTs that differ in one or more of the HLA α chains, HLA β chains, or peptides.

In additional embodiments, the stable multimers are attached to a solid support, such as a polymer, a flat surface, a bead, or a nanoparticle scaffold. In one non-limiting example, the solid support is a magnetic bead (such as Dynabeads). In some examples, a library including a plurality of solid supports (such as beads or nanoparticles) is provided, each including a different SCT multimer that is attached or linked to the support. In some embodiments, biotinylated SCT monomers or tetramers are incorporated onto a scaffold containing streptavidin, such as a streptavidin-coated bead or nanoparticle or a streptavidin-coated surface (such as a multi-well plate).

### IV. Methods of Use

Also disclosed herein are methods of using the disclosed MHC Class II SCTs. These methods include identifying an antigen-specific CD4+ T cell. In some embodiments, the methods further include identifying the T cell receptor (TCR) of the antigen-specific T cell, and in some examples, producing a population of T cells that express the identified TCR. In further embodiments, the population of T cells may be administered to a subject in need thereof.

In some embodiments, the methods include screening a population of T cells (e.g., contacting a population of T cells) with one or more stable multimers of a soluble human glycosylated MHC Class II SCT protein disclosed herein. In some examples, the population of T cells is contacted with a library of stable multimers, for example including a plurality of different SCT multimers, wherein each of the SCT multimers includes a different peptide sequence (such as a plurality of different peptide antigens and/or a plurality of HLA α/β combinations). This allows detection of one or more T cells in the population that are reactive to a particular peptide, which are referred to in some examples as “antigen-specific T cells.” In some examples, the T cells screened with the SCTs are produced from peripheral blood mononuclear cells (PBMC) stimulated with the peptides included in the plurality of the SCTs.

The reactive T cells in the population can be sorted and captured, for example using flow cytometry. In some examples, the reactive T cells are expanded in vitro using cell culture methods known to one of skill in the art. In some embodiments, the T cells are analyzed to identify the TCR expressed in the reactive cells. In one example, the TCR is sequenced, for example, using next generation sequencing methods (for example, bulk sequencing or 10X single-cell sequencing).

The identified TCR is cloned into an expression vector, and a population of T cells is transformed with the expression vector encoding the TCR, to produce a population of T cells (e.g., CD⁴ T cells) expressing the TCR. Methods of transforming T cells to express a heterologous protein (such as the identified TCR) are known to one of ordinary skill in the art. This population of transformed T cells may be administered to a subject in need thereof. Methods of adoptive cell transfer are known to one of ordinary skill in the art. In some examples, the T cells expressing the TCR are reactive to a tumor-associated antigen or a neoantigen, and are administered to a subject with cancer. In other examples, the T cells expressing the TCR are reactive to a viral or bacterial antigen and are administered to a subject infected with the virus or bacteria.

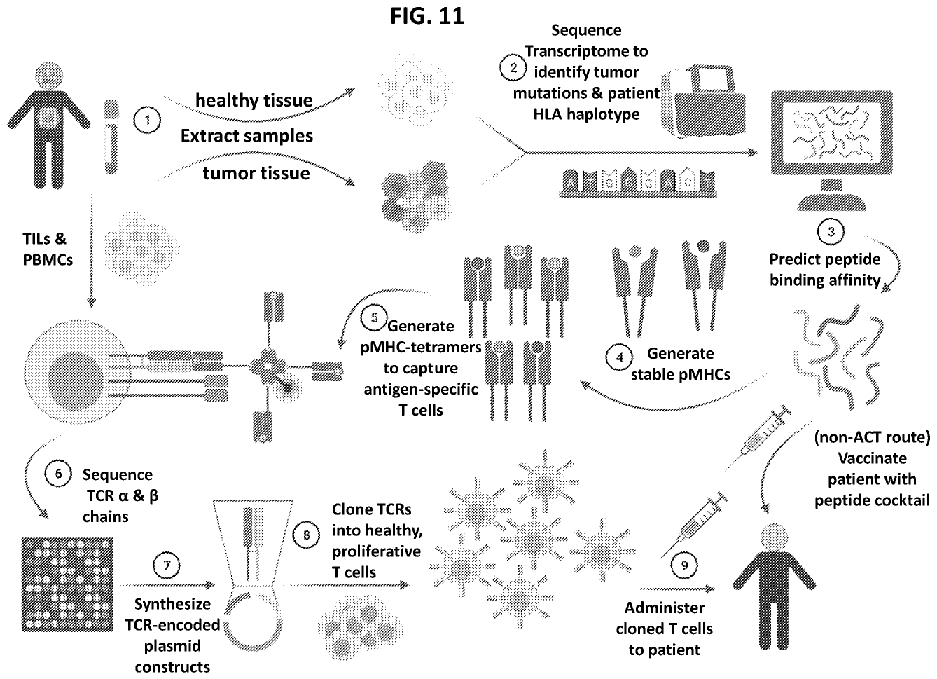

In some examples, the peptides used to generate the SCTs and screen the population of T cells are from a subject, such as a subject with cancer. In some examples, the population of T cells expressing the identified TCR are also from the subject (for example, are autologous T cells). A specific embodiment of the methods is illustrated in FIG. 11 and described in Example 5. However, one of ordinary skill in the art will recognize that modifications to these methods are possible.

### EXAMPLES

The following examples are provided to illustrate certain particular features and/or embodiments. These examples should not be construed to limit the disclosure to the particular features or embodiments described.

### Example 1

### Materials and Methods

SCT template production: The construction of plasmids was initiated by designing Class II SCT-encoded fragments to be inserted into a pcDNA3.1 vector for subsequent protein expression using the Expi293 transfection kit (Thermo Fisher Scientific). All ordered fragments (Twist Bioscience) were codon-optimized for human species protein expression according to Expi293 expression guidelines. The Zhu et al. fragment design (SCT-Z) consists of protein subunits encoded in the following order: secretion signal, peptide, peptide-β chain linker (L1), β chain, β-α chain linker (L2), α chain, AviTag, 6xHisTag (FIG. 2A). The Thayer et al. fragment design (SCT-T) consists of protein subunits encoded in the following order: secretion signal, α chain, α chain-invariant chain linker (L1), invariant chain fragment, peptide, peptide-β chain linker (L2), (3 chain, AviTag, 6xHisTag (FIG. 2B). For the SCT-Z designs, Gibson assembly overlaps (40 bp) were introduced within the initial 30 aa region of the post-signal sequence alpha chain for HLA alleles from each of the three loci (FIG. 3). HLA protein sequences at this region are invariant within each locus, which enables the ability to swap β and α chain of the first and second fragments of each construct, respectively, to generate new α/β pairs. For the SCT-T designs, the invariant chain region is a fixed sequence across all α/β pairs, so this part of the fragment encodes a Gibson assembly overlap (40 bp) to allow for HLA modularity (FIG. 4). Alternatively, the Bsu36I recognition site embedded within can be used to generate new α/β pairs by restriction enzyme digest (FIG. 4). The protein sequences of each HLA allele were obtained from an FTP server hosted by The Immuno Polymorphism Database (ftp.ebi.ac.uk/pub/databases/ipd/imgt/hla/fasta/). The peptide sequence derived from the invariant chain spans position 74-101 (UniProt reference number P04233, incorporated herein by reference as present in the database on Feb. 18, 2021). The mouse MHC alleles for I-Ab a and 13 chains were UniProt reference numbers P14434 and P14483, respectively (both incorporated herein by reference as present in the database on Feb. 18, 2021).

The ordered fragments were PCR-amplified by KOD Hot Start DNA Polymerase (Millipore Sigma) and paired together using NEBuilder® HiFi DNA Assembly Master Mix (New England Biolabs). The complete SCT-encoded fragment was subsequently double digested at the flanking regions by EcoRI and XhoI (New England Biolabs), and ligated into the MCS region of pcDNA3.1 vector. This process can be iterated for every unique α/β pairing under either Z or T designs to generate a template plasmid upon which additional molecular engineering steps are performed to substitute the encoded peptide for library production.

SCT peptide library production: Traditional PCR methods were implemented for substitution of peptides into SCT-Z constructs. Universal binding sites for reverse primer (peptide_sub.REV, 5′-AGCAAGAGCAAGAGGAG-3′; SEQ ID NO: 24) and forward primer (peptide_sub.FOR, 5′-GGTGGAGGAGGTTCTC-3′; SEQ ID NO: 25) were implemented into the regions upstream and downstream of the peptide, respectively. Reverse complement codons encoding the target antigen were appended onto the 5′ end of the reverse primer. Inverse PCR of the Class II SCT with a peptide-encoded reverse primer (peptide_sub.REV appended with peptide) and universal forward primer (peptide_sub.FOR), followed by treatment of the PCR product with

T4 DNA ligase, T4 polynucleotide kinase, and DpnI (New England Biolabs) allowed for re-construction of a plasmid with the replaced peptide (FIG. 5A). The plasmid was transformed into TOP10 chemically competent cells (Thermo Fisher Scientific).

An alternative method to substitute peptides was utilized for SCT-T designs. For any given peptide, a pair of primers were designed in which the first primer encodes the carboxy-terminal region of the invariant chain and the former half of the peptide, while the second primer encodes the latter half of the peptide and the amino-terminal region of L2. These primers are designed to overlap and bind together at the region where the middle of the peptide is encoded, such that PCR amplification will result in a dsDNA product encoding the terminal region of the invariant chain, the entirety of the peptide, and the initial region of L2. The invariant chain and L2 are fixed sequences across all templates, and include Bsu36I and BspEI restriction enzyme cut sites, respectively. Double digestion with these enzymes was conducted on both the template plasmid and the dsDNA PCR product; FIG. 5B). The desired products were gel-purified, ligated together, and transformed into TOP10 chemically competent cells.

SCT expression: Purified SCT plasmids were transfected into Expi293 cells within 24-well (2.5 ml capacity) plates. Briefly, 1.25 μg of plasmid was mixed with 75 μl Opti-MEM reduced serum media. 7.5 μl of ExpiFectamine™ Reagent was mixed with 70 μl Opti-MEM reduced serum media, incubated at room temperature for 5 minutes, and combined with the plasmid mixture. After a 15 minute room temperature incubation, the solution was added to 1.25 ml of Expi293 cells at 3 million cells/ml into a 24-well plate, which was then shaken at 225 RPM at 37° C. in 8% CO₂ overnight. Twenty hours later, a solution containing 7.5 μl of ExpiFectamine™ Transfection Enhancer 1 and 75 μl of ExpiFectamine™ Transfection Enhancer 2 was added to each well. The plate was kept on the shaker using aforementioned settings for a total of 4 days from start of transfection.

SCT biotinylation purification: On day 4 of transfection, Expi293 cells were pelleted to enable collection of the supernatant (containing secreted SCTs). An aliquot of this supernatant was saved for SDS-PAGE gel analysis. If the SCTs were to be used for functional assays, they were re-suspended into 20 mM bicine PBS buffer to allow for biotinylation, purified by HisTag column-loaded pipet tips in an MEA 2 automated purification system (PhyNexus), and subsequently desalted into PBS buffer using Zeba 7k MWCO columns. SCTs that were used for downstream experiments involving tetramerization were stored at −20° C. in PBS buffer with 20% glycerol. SCTs that were to be used for thermal stability measurements were instead stored at 4° C. in PBS buffer.

Thermal stability characterization: SYPRO™ Orange Protein Gel Stain was purchased from ThermoFisher Scientific and diluted with H₂O to give a 100X working solution. To each 19 aliquot of Class II SCT protein solution (diluted to ˜10 μM, if possible), 1 μl of the 100X dye solution was added. A Bio-Rad thermal cycler equipped with a CFX96 real-time PCR detection system was used in combination with Precision Melt Analysis software to obtain melting curves of each SCT sample. Thermal ramp settings were 25° C. to 95° C., 0.2° C. per 30 seconds.

Deglycosylation: Deglycosylation of SCTs was conducted with the PNGase F kit (New England Biolabs). Briefly, 20 μg of SCT was mixed with 1 μl of Glycoprotein Denaturing Buffer (10X) in a 10 μl H₂O solution. The mixture was denatured at 100° C. for 10 min, chilled on ice, and centrifuged for 10 seconds. To this solution, 2 μL GlycoBuffer 2 (10X), 2 μl 10% NP-40, and 6 μl H₂O was added. Then, 1 μl PNGase F was mixed, and the entire solution was incubated at 37° C. for 1 hour. For analysis of non-denatured proteins, 1 μl PNGase F was added directly to the 10 μl H₂O solution without the addition of the mix containing GlycoBuffer 2 and NP-40.

Antigen-specific CD4⁺ T cell isolation (Method 1): SCT-tetramer pool based. The monomer SCTs were individually tetramerized with PE or APC labeled streptavidin at a 4:1 molar ratio for 30 min at RT (or overnight at 4° C.). Biotin was added at an 8:1 molar ratio to streptavidin to block unoccupied biotin binding sites on streptavidin prior to mixing with the different tetramer samples. Each of the SCT-tetramers were pooled together and maintained at an individual tetramer concentration of 50 nM. The thawed 1M PBMCs then were re-suspended in complete R10 media supplemented with IL-2 (50 IU/ml) and incubated for overnight recovery. On the next day, the PBMCs were washed and incubated in PBS added with tyrosine kinase inhibitor (Dasatinib, 50 nM) for 30 min. The PBMCs were then stained with Annexin V-BV421 (1 μg/ml) and CD4-FITC antibody (1 μg/ml) for 10 min at 4° C. followed by incubation with a pool of SCT-tetramers (each, 20 nM). Antigen-specific CD4⁺ T cells captured by SCT-tetramer-PE were sorted into the tube using FACS sorter.

Antigen-specific CD4⁺ T cell isolation (Method 2): Peptide stimulation based. A vial containing 1 million peripheral blood mononuclear cells (PBMCs) were thawed and incubated in complete R10 media (500 ml of RPMI 1640; 50 ml heat-inactivated fetal bovine serum (FBS); 5 ml of Pen/Strep (100 U/ml penicillin and 100 ug/ml streptomycin); 1x GlutaMAX) by adding 1 μM of 15-mer peptide (or equivalent peptide pool, 15-mer with 11 overlap) and anti-CD40 antibody (1 μg/ml) for 16 hrs. On the next day, the cells were washed and stained with Annexin V-BV421 (1 μg/ml) and CD4-FITC antibody (1 μg/ml) and CD154-PE antibody (1 μg/ml) for 10 min at 4° C. Activation-induced expression of CD154 by peptide stimulation permits the sorting of antigen-specific T-cells expressing these biomarkers.

Antigen-specific T cell functional assay (Method 1): SCT-Tetramer based. A vial of 0.5 M antigen-specific CD4+ T cells suspended in serum-free cell culture media (CTL-Test™ Medium, Immunospot) was stimulated with SCT tetramers (0.1 μM final concentration) at 37° C. After 16 hours of incubation, the supernatant of the cell solution was extracted for analysis by standard ELISA protocols for TNF-α (RD Systems, DY210-05), IFNγ (RDSystems, DY285B-05), and IL-2 (BioLegend, 431804).

Antigen-specific T cell functional assay (Method 2): Peptide stimulation based. 10⁵-10⁶ PBMCs or cloned CD4⁺ T cells were incubated in 100 μL of serum-free cell culture media (CTL-Test™ Medium, Immunospot) by adding 1 μM of 15-mer peptide (or equivalent peptide pool, 15-mer with 11 overlap) for 16 hrs. After 16 hours of incubation, the supernatant of the cell solution was extracted for analysis by standard ELISA protocols for TNF-α (RD Systems, DY210-05), IFN-γ (RDSystems, DY285B-05), and IL-2 (BioLegend, 431804).

CD4⁺ T cell expansion: The FACS-sorted antigen-specific CD4⁺ T cells were directly transferred to Rapid Expansion Protocol (REP) media (Ho et al., J. Immunological Meth. 310:40-52, 2006). REP media is composed of 2.5M irradiated PBMCs (4000 RAD), 0.5M TM-LCL (8000 RAD), IL-2 (50 IU/ml) and anti-CD3 antibody (30 ng/ml) per ml. On Day 3, half of the medium was removed without disturbing cells, and replaced with an equivalent volume of REP medium and cytokines. On Days 6-12, this media replacement step was repeated approximately every 2 days.

On Day 14, cells were sorted with SCT tetramers to isolate the antigen-specific T cell populations. Multiple aliquots of cells were either frozen down or REP cycles were repeated to continue expansion, if needed for further analyses.

Sorting and TCR sequencing of expanded T cells: The expanded CD4+ T cells were analyzed by flow cytometry using SCT-tetramers to enumerate individual antigen-specific T cell populations. 100,000 CD4⁺ T cell were stained with Annexin V-BV421 (1 μg/ml) and CD4-FITC antibody (1 μg/ml) for 10 min at 4° C., followed by incubation with three tetramers with different dyes (PE, PE/Cy7, and APC) at 20 nM. The frequency of antigen-specific T cell population was measured by FlowJo software. The sorted cells were collected by antigen specificity in a 96-well plate, lysed, and RT-PCR was conducted to amplify the TCR α/β chains. The PCR product was extended with sequencing primers and the library was analyzed by Miseq software to extract antigen-paired TCR sequencing data. The sequencing data was further analyzed by customized R code and MIXCR.

### Example 2

### Generation and Characterization of SCTs

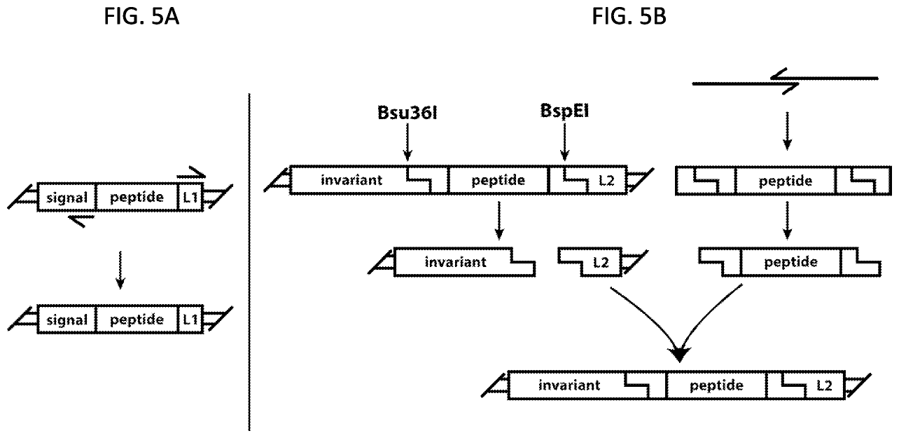

To demonstrate viable SCT expression under these two designs, five initial SCTs were constructed (Table 1). The first plasmid used an SCT-T template with the Ea (52-68) peptide (ASFEAQGALANIAVDKA; SEQ ID NO: 19) and mouse MHC alleles IAb α/β. This sample was transfected as a biological triplicate to demonstrate reproducibility of the transfection method (FIG. 6A). Another four plasmids, designed under the SCT-Z template for the HLA allele pair DRA*01:01/DRB1*01:01, consisted of a small library of peptides selected from citations identified through IEDB.org (Strug et al., J. Proteome Res. 7:2703-2711, 2008; Kwok et al., J. Immunol. 188:2537-2544, 2012; Galperin et al., Science Immunology 3(24), 2018). A positive control SCT consisting of a Class I SCT encoding HLA-A*02:01 with the WT1 (RMFPNAPYL; SEQ ID NO: 8) peptide was used to confirm transfection and to indicate the expected size for a Class I pMHC construct (approximately 50 kDa). The first Class II SCT plasmid produced a mass of approximately 60 kDa as previously reported, and all three biological replicates showed high and consistent yield. For the SCT-Z plasmids, the detected masses were also within the same range, but the last sample, interestingly, revealed a larger mass, which was attributed to the presence of an additional glycan group on this pMHC, given the presence of the NQT glycosylation motif within its peptide sequence (APIYNVLPTTSLVLGKNQTL; SEQ ID NO: 23). Furthermore, the expression yield of SCTs based on the Zhu et al. design appeared to be peptide-dependent, implying that the degree of stabilization afforded by the selected peptide has a significant impact on expression level of the SCT construct.

TABLE 1
Exemplary SCT components

| Well | SCT | | MHC α | MHC β |
|---|---|---|---|---|
| ID | type | Peptide | chain | chain |
| 1 | T | ASFEAQGALANIAVDKA | I-Ab α | I-Ab β |
| 2 | | (SEQ ID NO: 19) | | |
| 3 | | | | |
| 4 | Z | RFYKTLRAEQASQ | HLA- | HLA- |
| | | (SEQ ID NO: 20) | DRA*01:01 | DRBl*01:01 |
| 5 | | SMRYQSLIPRLVEFF | | |
| | | (SEQ ID NO: 21) | | |
| 6 | | VGSDWRFLRGYHQYA | | |
| | | (SEQ ID NO: 22) | | |
| 7 | | APIYNVLPTTSLVLGKNQTL | | |
| | | (SEQ ID NO: 23) | | |

Thermal shift assay measurements of the five plasmids were performed to assess stability. As seen in FIG. 6B, regardless of the template or peptide used, melting temperature (Tm) values based upon the absolute minimum of the derivative for fluorescence over temperature was approximately the same (70-75° C.).

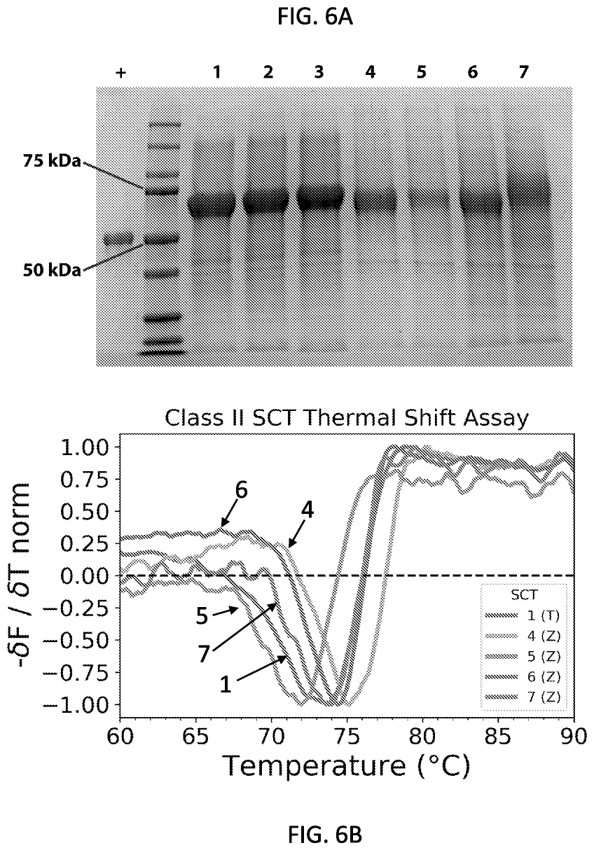

For SCT-T and SCT-Z designs, the expected masses were 55 kDa and 51 kDa, respectively. However, the observed SCT masses were closer to the 75 kDa marker of the protein ladder (FIG. 6A). This was suspected to be a result of glycosylation. Deglycosylation of SCTs from both designs using PNGase treatment demonstrated a similar extent of decreased mass, closer to the calculated range of 51-55 kDa (FIG. 7).

### Example 3

### Functional Validation Assays

Based upon characterization assays indicating higher yield and perhaps improved stability of SCT-T compared to SCT-Z, SCT-T was chosen as the template for performing downstream functional validation assays. Toward this end, an SCT-T template was rebuilt to encode human MHC (HLA-DRA*01:01/DRB1*01:01), and a previously identified influenza virus peptide (PKYVKQNTLKLAT; SEQ ID NO: 9) was inserted, as well as an irrelevant peptide (TRFQTLLALHRSYLT; SEQ ID NO: 26), from SARS-CoV-2 spike protein.

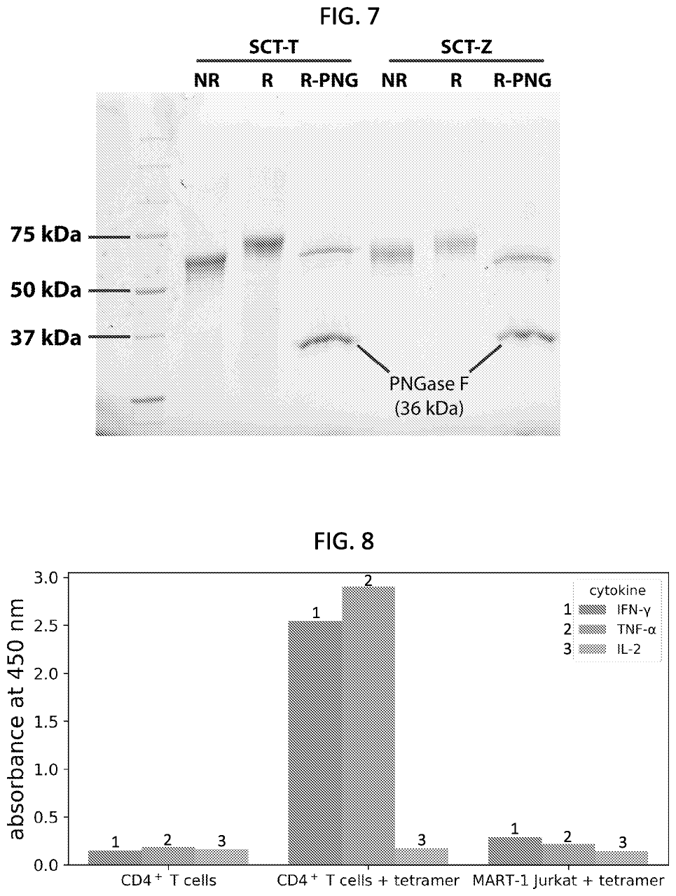

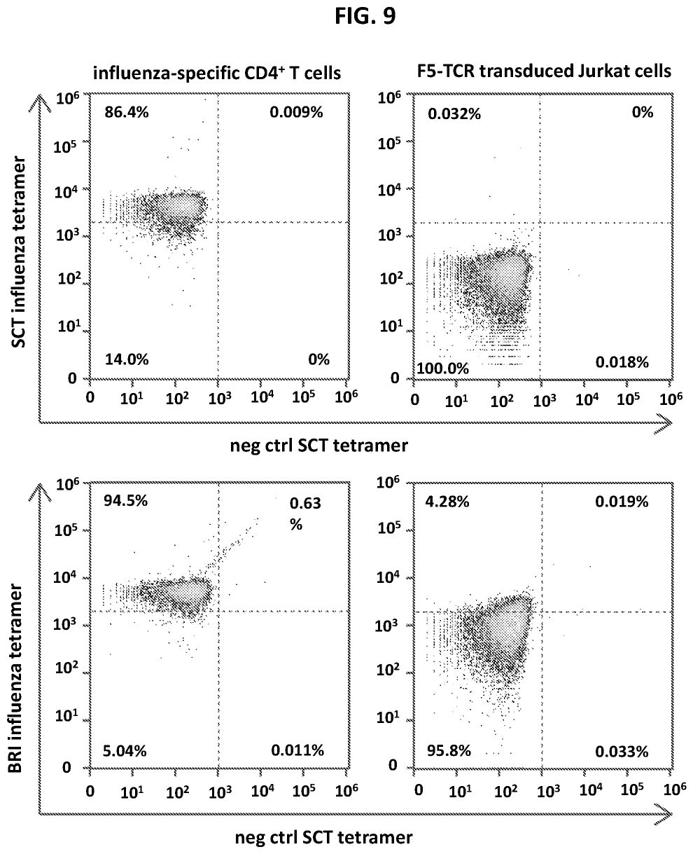

A CD4⁺ T cell line specific to the influenza peptide and cognate pMHC-tetramers (produced by traditional methods requiring exogenous loading of peptide) were purchased from Benaroya Institute (BRI) to validate the SCT tetramers. Peptide specificity of the CD4⁺ T cell line was confirmed via ELISA cytokine assay following overnight stimulation using influenza SCT tetramers (FIG. 8), which showed a significant increase in IFN-γ and TNF-α secretion only from the CD4⁺ T cells. Flow cytometry assays indicated that the SCT tetramers performed similarly to the BRI tetramer variants in terms of binding sensitivity with the influenza-specific CD4⁺ T cell line (FIG. 9). When both tetramer variants were incubated with a Jurkat cell line expressing an irrelevant TCR, however, the SCT tetramers showed significantly less binding, indicating reduced non-specific binding.

Taken together, the class II SCT variants demonstrated similar capabilities as BRI variants in terms of binding to cognate TCRs, and appear to be “cleaner” reagents as they appear to have less cross-reactivity. This difference may be due to the fact that SCTs undergo intracellular packaging and so can make use of native folding mechanisms, whereas for BRI variants, the class II a and chains were co-expressed, extracted as an empty MHC construct, and then exogenously loaded with peptide. This additional step required for BRI tetramers may cause higher susceptibility to misfolding that could explain the increased non-specificity.

### Example 4

### High Throughput Identification of Antigen Specificity and TCR Sequences

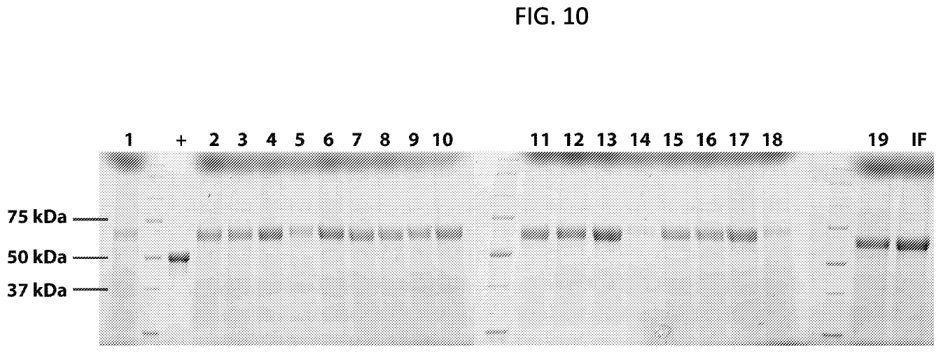

Recently, CD4⁺ T cells have been highlighted in numerous studies for their immunoprotective role against SARS-CoV-2. In order to better understand the functional role played by these T cells, we wanted to identify their antigen specificity and cognate TCR sequences. Toward this end, 19 peptide candidates were identified from SARS-CoV-2 structural proteins demonstrated by others to be immunogenic in patients with the paired HLA-DRA*01:01/DRB1*01:01 alleles (Table 2). These peptides were substituted into the SCT-T template for transfection into Expi293 cells. Eighteen of the plasmids resulted in detectable SCT protein expression (FIG. 10), although to varying degrees. Peptide 14, corresponding to an envelope protein epitope, resulted in no expression. For the eighteen SCTs that were expressed, they were individually tetramerized and then pooled together.

TABLE 2
Peptides selected for assembly into SCT-T template plasmids

| Well | | | Affinity | SEQ ID |
|---|---|---|---|---|
| ID | Peptide | Antigen | (nM) | NO: |
| 1 | CTFEYVSQPFLMDLE* | SP_166-180 | 22.5 | 27 |
| 2 | ITRFQTLLALHRSYL^{∧} | SP_23 5-249 | 8.6 | 28 |
| 3 | TRFQTLLALHRSYLT# | SP_236-250 | 7.5 | 29 |
| 4 | FNFNGLTGTGVLTES# | SP_541-555 | 9.3 | 30 |
| 5 | NLLLQYGSFCTQLNR*# | SP_751-765 | 64.6 | 31 |
| 6 | TQLNRALTGIAVEQD# | SP_761-775 | 9.7 | 32 |
| 7 | CAQKFNGLTVLPPLL# | SP_851-865 | 14 | 33 |
| 8 | TDEMIAQYTSALLAG* | SP_866-880 | 17.8 | 34 |
| 9 | WTFGAGAALQIPFAM# | SP_886-900 | 6.1 | 35 |
| 10 | IPFAMQMAYRFNGIG# | SP_896-910 | 9.8 | 36 |
| 11 | TLVKQLSSNFGAISS# | SP 961-975 | 8.3 | 37 |
| 12 | VQIDRLITGRLQSLQ# | SP_991-1005 | 11.3 | 38 |
| 13 | LITGRLQSLQTYVTQ# | SP_996-1010 | 6.1 | 39 |
| 14 | LAILTALRLCAYCCN# | E_31-45 | 16.8 | 40 |
| 15 | SWFTALTQHGKEDLK% | N_51-65 | 21.9 | 41 |
| 16 | KDGIIWVATEGALNT^{∧} | N_127-141 | 10 | 42 |
| 17 | AIVLQLPQGTTLPKG^{∧} | N_156-170 | 25 | 43 |
| 18 | GAVILRGHLRIAGHHLGR* | M_141-158 | 26.1 | 44 |
| 19 | PKEITVATSRTLSYYKL* | M165-181 | 23.4 | 45 |
| IF | PKYVKQNTLKLAT | HA 306-318 | 116.7 | 9 |

*Peng et al., Nature Immunology 21:1336-1345, 2020; ^{∧}Nelde et al., Nature Immunology 22:74-85, 2021; #Mateus et al., Science 370:89-94, 2020; %Le Bert et al., Nature 584:457-462, 2020

Four DRB1*01:01-positive patient PBMC samples from an in-house bio-specimen bank were thawed, stimulated overnight with peptides matching the sequences of those used in the SCTs, and sorted for CD4⁺ T cells the following day. The pooled tetramers were then used to capture and sort SCT-specific CD4⁺ T cells. The sorted cells were subsequently expanded for approximately two weeks (Ho et al., J. Immunological Meth. 310:40-52, 2006). Flow cytometry of the expanded cells using individual SCT tetramers revealed variable degrees of specificity to each of the 18 elements. The CD4+ T cells, now individually sorted by antigen specificity, were submitted for NGS bulk sequencing to identify TCR sequences. The TCRs were cloned into CD4⁺ T cells and shown to bind to the correct tetramer, demonstrating that they were capable of recognizing the SCT-presented epitope. To confirm that they were capable of recognizing biologically-presented epitope, APCs pulsed with the immunogenic peptides were incubated with the T cells. Flow assays confirmed surface level and intracellular expression of activation/inflammation marker proteins, while ELISA assays demonstrated a significant increase of activation/inflammatory protein secretion when the correct peptide was introduced to each cell line.

### Example 5

### Adoptive Transfer Cell Therapy

This example describes methods that can be used to produce a population of T cells expressing an antigen-specific T cell receptor and administering the cells to a subject. While particular methods are provided, one of skill in the art will recognize that methods that deviate from these specific methods can also be used, including addition or omission of one or more steps.

An exemplary method for identifying antigen-specific T cell receptors from a subject, such as a subject with a tumor and administering a population of T cells expressing the TCRs to the subject is schematically illustrated in FIG. 11. Healthy (non-tumor) tissue and tumor tissue is extracted and analyzed by sequencing of the transcriptome to identify neoantigens and also the HLA haplotype of the subject. Peptide-MHC binding affinity predictions are performed to identity the best peptide candidates of the neoantigen for pMHC generation. Stable pMHCs are then produced and tetramerized as described herein. These are used to capture antigen-specific T cells. TCRs from the captured T cells are sequenced and synthesized in plasmid expression constructs. These are transformed into healthy T cells and administered to the subject by adoptive cell therapy protocols. In some examples, the antigen-specific T cells, the transformed T cells, or both are from the subject being treated, but in other examples, one or both could be from another subject.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A nucleic acid fragment pair comprising a first nucleic acid fragment and second nucleic acid fragment that, when assembled, encode a major histocompatibility complex (MHC) Class II single chain trimer (SCT) protein, the SCT comprising as operably linked subunits a human leukocyte antigen (HLA) alpha chain, an HLA beta chain, and a peptide, and wherein the first nucleic acid fragment and the second nucleic acid fragment each comprise a portion of an assembly site in a position, when assembled, encoding an invariant region separating the HLA alpha chain and the HLA beta chain of the encoded MHC Class II SCT protein.

2. The nucleic acid fragment pair of claim 1, wherein the assembly site is a Gibson assembly site.

3. The nucleic acid fragment pair of claim 1, wherein the MHC Class II SCT protein encoded by the assembled nucleic acid fragment pair comprises protein subunits encoded in the following order:
secretion signal, HLA alpha chain extracellular domain, HLA alpha chain-invariant chain linker (L1), invariant region, peptide, peptide-HLA beta chain linker (L2), HLA beta chain extracellular domain, and optionally, one or more purification tags, and wherein the assembly site is positioned within the invariant region; or
secretion signal, peptide, peptide-HLA beta chain linker (L1), HLA beta chain extracellular domain, HLA beta-alpha chain linker (L2), HLA alpha chain extracellular domain, and optionally, one or more purification tags, and wherein the assembly site is positioned within an invariant region of the HLA alpha chain.

4. (canceled)

5. The nucleic acid fragment pair of claim 3, wherein:
the secretion signal is selected from an HLA secretion signal, an interferon-α2 secretion signal, and an interferon-γ secretion signal; and/or the peptide is an antigen peptide, a self peptide, or a placeholder peptide.

6-9. (canceled)

10. The nucleic acid fragment pair of claim 1, wherein the nucleic acid fragment pair is codon-optimized for mammalian expression.

11. A nucleic acid molecule comprising the assembled nucleic acid fragment pair of claim 1, wherein the assembled nucleic acid fragment pair comprises the first nucleic acid fragment operably linked to the second nucleic acid fragment.

12. A vector comprising the nucleic acid molecule of claim 11.

13-14. (canceled)

15. A human cell line transformed with the vector of claim 12.

16-17. (canceled)

18. A library comprising a plurality of the nucleic acid fragment pairs of claim 1.

19. A library comprising a plurality of the assembled nucleic acid fragment pairs of claim 18.

20. A human-glycosylated MHC Class II single chain trimer (SCT) protein.

21. The human-glycosylated MHC Class II SCT protein of claim 20, wherein the SCT protein is soluble.

22. The soluble human-glycosylated MHC Class II SCT protein of claim 21, comprising an antigen peptide, a self peptide, or a placeholder peptide.

23. (canceled)

24. The soluble human-glycosylated MHC Class II SCT protein of claim 21, comprising:
(a) an HLA alpha chain extracellular domain, an HLA alpha chain-invariant chain linker (L1), an invariant chain, a peptide, a peptide-HLA beta chain linker (L2), and an HLA beta chain extracellular domain, in N-terminal to C-terminal order; or
(b) a peptide, a peptide-HLA beta chain linker (L1), an HLA beta chain extracellular domain, an HLA beta-alpha chain linker (L2), and an HLA alpha chain extracellular domain.

25-26. (canceled)

27. The soluble human-glycosylated MHC Class II SCT protein of claim 21, wherein the SCT protein is assembled as a stable multimer.

28. The soluble human-glycosylated MHC Class II SCT protein of claim 27, wherein the stable multimer is a tetramer.

29. The soluble human-glycosylated MHC Class II SCT protein of claim 27, wherein the stable multimer is attached to a polymer or a nanoparticle scaffold.

30. A library comprising a plurality of soluble human-glycosylated MHC Class II SCT proteins of claim 21.

31. A library comprising a plurality of stable multimers of claim 27.

32. A method of identifying an antigen-specific CD4⁺ T cell, comprising:
contacting a T cell population with one or more of the stable multimers of a soluble human glycosylated MHC Class II SCT protein of claim 27; and
identifying a CD4⁺ T cell reactive thereto.

33. The method of claim 32, further comprising:
sequencing the T cell receptor (TCR) of the identified antigen-specific CD4⁺ T cell; and
producing a population of T cells expressing the antigen-specific TCR.

34. The method of claim 33, further comprising administering the population of T cells expressing the antigen-specific TCR to a subject in need thereof.

35. (canceled)


SEQUENCE LISTING (60 sequences)

SEQ ID NO: 1 - DNA, 1377 bp, Artificial Sequence
  SCT-Z design nucleic acid
atggtgtgtctgaagctgcccggaggcagctgcatgacagccctgacagtgaccctgatggtgctgagctctcctcttgctcttgcttcagcattatcagaaggagccaccccacaagatttaaacaccatgctaggtggaggaggttctctagtgccacggggctctggaggtggtggaagcggcgataccagacctagatttctgtggcagctgaaatttgagtgccacttctttaatggcaccgagagagtgagactgctggagagatgcatctataatcaggaagaaagcgtgagatttgatagcgatgtgggagaatatagagctgtgaccgaactgggaagacccgatgccgagtattggaacagccagaaggatctgctggaacagagaagagctgccgtggatacatactgtagacataattacggcgtgggagaaagctttacagtgcagagaagagtggaacctaaagtgacagtgtaccctagcaagacacagcccctgcagcatcacaatctgctggtgtgtagcgtgagcggattctatcctggaagcatcgaagtgagatggtttagaaacggccaggaagagaaggctggagtggtgagcacaggactgatccagaatggagattggacatttcagacactggtgatgctggaaacagtgcctagaagcggcgaggtgtatacatgtcaggtggaacatccaagcgtgacaagcccactgacagtggaatggagagctagatctgaatctgcacagagcaagggtggcggtgggagtggtggtggcggaatcaaggaagaacacgtgattattcaggctgagttctatctgaaccctgaccagagcggagagtttatgtttgactttgacggcgacgaaatttttcacgtggacatggctaagaaggagacagtgtggagactggaggagtttggaagatttgctagctttgaagctcagggagctctggctaatatcgctgtggataaggccaatctggagattatgaccaagaggagcaactacacacctatcaccaacgtgcctcctgaagtgacagtgctgacaaattctcctgtggagctgagagaacctaatgtgctgatctgcttcatcgataagttcaccccaccagtggttaatgtgacatggctgaggaatggcaagccagtgacaacaggagtgtctgaaacagtgtttctgcctagagaagaccacctgttcagaaagttccactatctgcctttcctgccctctacagaggatgtgtatgattgcagagtggaacattggggcctggatgagcctctgctgaaacactgggagtttgatgctccttctcctctgcctgagacaacagaa

SEQ ID NO: 2 - protein, 459 aa, Artificial Sequence
  SCT-Z design amino acid sequence
MVCLKLPGGSCMTALTVTLMVLSSPLALASALSEGATPQDLNTMLGGGGSLVPRGSGGGGSGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKGGGGSGGGGIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTE

SEQ ID NO: 3 - DNA, 1450 bp, Artificial Sequence
  SCT-T design nucleic acid sequence
atggctattagtggagtgcccgtgctgggcttcttcatcatcgccgtgctgatgagcgcccaggagagctgggccatcaaggaagaacacgtgattattcaggctgagttctatctgaaccctgaccagagcggagagtttatgtttgactttgacggcgacgaaatttttcacgtggacatggctaagaaggagacagtgtggagactggaggagtttggaagatttgctagctttgaagctcagggagctctggctaatatcgctgtggataaggccaatctggagattatgaccaagaggagcaactacacacctatcaccaacgtgcctcctgaagtgacagtgctgacaaattctcctgtggagctgagagaacctaatgtgctgatctgcttcatcgataagttcaccccaccagtggttaatgtgacatggctgaggaatggcaagccagtgacaacaggagtgtctgaaacagtgtttctgcctagagaagaccacctgttcagaaagttccactatctgcctttcctgccctctacagaggatgtgtatgattgcagagtggaacattggggcctggatgagcctctgctgaaacactgggagtttgatgctccttctcctctgcctgagacaacagaaggtggcggtggatctggtggcggagggtctcagcagggaagactggataagctgacagtgactagccagaacctgcagctggaaaacctgaggatgaagctgcctaaacctccatcagcattatcagaaggagccaccccacaagatttaaacaccatgctaggtggaggaagttcaggaggtggtggaagcggtggtggcgggtctggcgataccagacctagatttctgtggcagctgaaatttgagtgccacttctttaatggcaccgagagagtgagactgctggagagatgcatctataatcaggaagaaagcgtgagatttgatagcgatgtgggagaatatagagctgtgaccgaactgggaagacccgatgccgagtattggaacagccagaaggatctgctggaacagagaagagctgccgtggatacatactgtagacataattacggcgtgggagaaagctttacagtgcagagaagagtggaacctaaagtgacagtgtaccctagcaagacacagcccctgcagcatcacaatctgctggtgtgtagcgtgagcggattctatcctggaagcatcgaagtgagatggtttagaaacggccaggaagagaaggctggagtggtgagcacaggactgatccagaatggagattggacatttcagacactggtgatgctggaaacagtgcctagaagcggcgaggtgtatacatgtcaggtggaacatccaagcgtgacaagcccactgacagtggaatggagagctagatctgaatctgcacagagcaaga

SEQ ID NO: 4 - protein, 483 aa, Artificial Sequence
  SCT-T design amino acid sequence
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEGGGGSGGGGSQQGRLDKLTVTSQNLQLENLRMKLPKPPSALSEGATPQDLNTMLGGGSSGGGGSGGGGSGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 5 - DNA, 57 bp, Artificial Sequence
  SCT-Z design invariant region portion
atcaaggaagaacacgtgattattcaggctgagttctatctgaaccctgaccagagc

SEQ ID NO: 6 - protein, 19 aa, Artificial Sequence
  SCT-Z design invariant region portion
IKEEHVIIQAEFYLNPDQS

SEQ ID NO: 7 - protein, 62 aa, Artificial Sequence
  SCT-T design invariant chain, peptide, and linker portion
GGSQQGRLDKLTVTSQNLQLENLRMKLPKPPSALSEGATPQDLNTMLGGGSSGGGGSGGGGS

SEQ ID NO: 8 - protein, 9 aa, Artificial Sequence
  WT1 peptide
RMFPNAPYL

SEQ ID NO: 9 - protein, 13 aa, Artificial Sequence
  Influenza peptide
PKYVKQNTLKLAT

SEQ ID NO: 10 - protein, 10 aa, Artificial Sequence
  MART-1 peptide
ELAGIGILTV

SEQ ID NO: 11 - protein, 15 aa, Artificial Sequence
  Biotin ligase site
GLNDIFEAQKIEWHE

SEQ ID NO: 12 - protein, 16 aa, Artificial Sequence
  Linker sequence
GGGGSLVPRGSGGGGS

SEQ ID NO: 13 - protein, 8 aa, Artificial Sequence
  Linker sequence
GGGGSGGG

SEQ ID NO: 14 - protein, 10 aa, Artificial Sequence
  Linker sequence
GGGGSGGGGS

SEQ ID NO: 15 - protein, 15 aa, Artificial Sequence
  Linker sequence
GGGSSGGGGSGGGGS

SEQ ID NO: 16 - protein, 9 aa, Artificial Sequence
  Linker sequence
TRGGASGGG

SEQ ID NO: 17 - protein, 28 aa, Artificial Sequence
  SCT-T design invariant region
QQGRLDKLTVTSQNLQLENLRMKLPKPP

SEQ ID NO: 18 - protein, 16 aa, Artificial Sequence
  Placeholder peptide
SALSEGATPQDLNTML

SEQ ID NO: 19 - protein, 17 aa, Artificial Sequence
  Peptide antigen
ASFEAQGALANIAVDKA

SEQ ID NO: 20 - protein, 13 aa, Artificial Sequence
  Peptide antigen
RFYKTLRAEQASQ

SEQ ID NO: 21 - protein, 15 aa, Artificial Sequence
  Peptide antigen
SMRYQSLIPRLVEFF

SEQ ID NO: 22 - protein, 15 aa, Artificial Sequence
  Peptide antigen
VGSDWRFLRGYHQYA

SEQ ID NO: 23 - protein, 20 aa, Artificial Sequence
  Peptide antigen
APIYNVLPTTSLVLGKNQTL

SEQ ID NO: 24 - DNA, 17 bp, Artificial Sequence
  Synthetic oligonulceotide primer
agcaagagcaagaggag

SEQ ID NO: 25 - DNA, 16 bp, Artificial Sequence
  Synthetic oligonucleotide primer
ggtggaggaggttctc

SEQ ID NO: 26 - protein, 15 aa, Artificial Sequence
  SARS-CoV-2 spike protein peptide
TRFQTLLALHRSYLT

SEQ ID NO: 27 - protein, 15 aa, Artificial Sequence
  SARS-CoV-2 peptide
CTFEYVSQPFLMDLE

SEQ ID NO: 28 - protein, 15 aa, Artificial Sequence
  SARS-CoV-2 peptide
ITRFQTLLALHRSYL

SEQ ID NO: 29 - protein, 15 aa, Artificial Sequence
  SARS-CoV-2 peptide
TRFQTLLALHRSYLT

SEQ ID NO: 30 - protein, 15 aa, Artificial Sequence
  SARS-CoV-2 peptide
FNFNGLTGTGVLTES

SEQ ID NO: 31 - protein, 15 aa, Artificial Sequence
  SARS-CoV-2 peptide
NLLLQYGSFCTQLNR

SEQ ID NO: 32 - protein, 15 aa, Artificial Sequence
  SARS-CoV-2 peptide
TQLNRALTGIAVEQD

SEQ ID NO: 33 - protein, 15 aa, Artificial Sequence
  SARS-CoV-2 peptide
CAQKFNGLTVLPPLL

SEQ ID NO: 34 - protein, 15 aa, Artificial Sequence
  SARS-CoV-2 peptide
TDEMIAQYTSALLAG

SEQ ID NO: 35 - protein, 15 aa, Artificial Sequence
  SARS-CoV-2 peptide
WTFGAGAALQIPFAM

SEQ ID NO: 36 - protein, 15 aa, Artificial Sequence
  SARS-CoV-2 peptide
IPFAMQMAYRFNGIG

SEQ ID NO: 37 - protein, 15 aa, Artificial Sequence
  SARS-CoV-2 peptide
TLVKQLSSNFGAISS

SEQ ID NO: 38 - protein, 15 aa, Artificial Sequence
  SARS-CoV-2 peptide
VQIDRLITGRLQSLQ

SEQ ID NO: 39 - protein, 15 aa, Artificial Sequence
  SARS-CoV-2 peptide
LITGRLQSLQTYVTQ

SEQ ID NO: 40 - protein, 15 aa, Artificial Sequence
  SARS-CoV-2 peptide
LAILTALRLCAYCCN

SEQ ID NO: 41 - protein, 15 aa, Artificial Sequence
  SARS-CoV-2 peptide
SWFTALTQHGKEDLK

SEQ ID NO: 42 - protein, 15 aa, Artificial Sequence
  SARS-CoV-2 peptide
KDGIIWVATEGALNT

SEQ ID NO: 43 - protein, 15 aa, Artificial Sequence
  SARS-CoV-2 peptide
AIVLQLPQGTTLPKG

SEQ ID NO: 44 - protein, 18 aa, Artificial Sequence
  SARS-CoV-2 peptide
GAVILRGHLRIAGHHLGR

SEQ ID NO: 45 - protein, 17 aa, Artificial Sequence
  SARS-CoV-2 peptide
PKEITVATSRTLSYYKL

SEQ ID NO: 46 - protein, 12 aa, Artificial Sequence
  Influenza A peptide
GPLKAEIAQRLE

SEQ ID NO: 47 - protein, 12 aa, Artificial Sequence
  Influenza A peptide
NFWRGENGRKTR

SEQ ID NO: 48 - protein, 13 aa, Artificial Sequence
  Influenza A peptide
DTIIFEANGNLIA

SEQ ID NO: 49 - protein, 13 aa, Artificial Sequence
  Influenza A peptide
EIIRMMESARPED

SEQ ID NO: 50 - protein, 13 aa, Artificial Sequence
  Influenza A peptide
ELLVLLENERTLD

SEQ ID NO: 51 - protein, 13 aa, Artificial Sequence
  Influenza A peptide
GPLKAEIAQRLED

SEQ ID NO: 52 - protein, 14 aa, Artificial Sequence
  Influenza A peptide
GQISIQPTFSVQRN

SEQ ID NO: 53 - protein, 20 aa, Artificial Sequence
  Bacillus anthracis peptide
APIYNVLPTTSLVLGKNQTL

SEQ ID NO: 54 - protein, 13 aa, Artificial Sequence
  Clostridium peptide
ELIHVLHGLYGMQ

SEQ ID NO: 55 - protein, 13 aa, Artificial Sequence
  Clostridium peptide
EVYKLVKAKWLGT

SEQ ID NO: 56 - protein, 16 aa, Artificial Sequence
  HIV peptide
PEVIPMFSALSEGATP

SEQ ID NO: 57 - protein, 15 aa, Artificial Sequence
  Vaccinia virus peptide
SMRYQSLIPRLVEFF

SEQ ID NO: 58 - protein, 20 aa, Artificial Sequence
  Human IDH1 peptide
GWVKPIIIGHHAYGDQYRAT

SEQ ID NO: 59 - protein, 15 aa, Artificial Sequence
  Human HLA class II antigen gamma chain peptide
KMRMATPLLMQALPM

SEQ ID NO: 60 - protein, 15 aa, Artificial Sequence
  Human collagen alpha-1 (II) chain peptide
GIAGFAGEQGPKGEP